(19) United States
(12) Patent Application Publication
(10) Pub. No.: US 2024/0254139 A1
(43) Pub. Date: Aug. 1, 2024

(54) FUSED ISOXAZOLYL COMPOUNDS AS KAT6A INHIBITORS

(71) Applicant: Aurigene Oncology Limited, Bangalore (IN)

(72) Inventors: Chandregowda Venkateshappa, Bangalore (IN); Chandrasekhar Abbineni, Hyderabad (IN); Susanta Samajdar, Bangalore (IN); Kalisankar Bera, Bangalore (IN)

(21) Appl. No.: 18/562,758

(22) PCT Filed: May 21, 2022
(86) PCT No.: PCT/IB22/54771

§ 371 Date: Nov. 20, 2023

(30) Foreign Application Priority Data
May 21, 2021 (IN) 202141022736

(51) Int. Cl. C07D 498/04; A61K 31/437; A61K 31/4545; A61K 31/4985; A61K 31/519; A61K 31/5377

(52) U.S. Cl. CPC C07D 498/04; A61K 31/437; A61K 31/4545; A61K 31/4985; A61K 31/519; A61K 31/5377

(57) ABSTRACT

The present invention provides fused isoxazolyl compound of formula (I), which are therapeutically useful as KAT6A inhibitors particularly in the treatment and/or prevention of diseases or disorders dependent upon KAT6A in a mammal. The present invention also provides preparation of the compounds and pharmaceutical compositions comprising at least one of the compound represented by formula (I) or a pharmaceutically acceptable salt or a stereoisomer or a tautomer thereof.

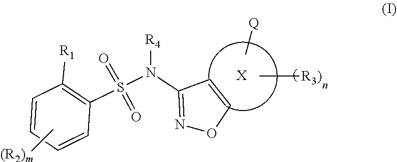

### RELATED APPLICATION

This application claims a benefit of Indian provisional application number 202141022736, filed on 21 May 2021; the specification of which is hereby incorporated by reference in their entirety.

### FIELD OF THE INVENTION

The present invention relates to fused isoxazolyl compounds and, a pharmaceutically acceptable salt or a stereoisomer or a tautomer thereof which are useful as KAT6A inhibitors and, for the treatment of diseases or disorders dependent upon or mediated by KAT6A. The present invention also relates to a method of preparation of the said isoxazolyl compounds and pharmaceutical compositions comprising the said compounds.

### BACKGROUND OF THE INVENTION

The MYST family of histone acetyltransferases (HATs) is named after its four founding members MOZ, Ybf2 (Sas3), Sas2, and Tip60. Presence of zinc fingers and chromodomains is characteristic feature of these HATs. MYST acetylates lysine residues on histones H2A, H3, and H4. Several MYST family proteins contain zinc fingers as well as the highly conserved motif A found among GNATs that facilitates acetyl-CoA binding. MYST HATs are involved in a number of key nuclear processes and play critical roles in gene-specific transcription regulation, DNA damage response, repair and replication. The anomalous activity of these HATs or their associated complexes can easily lead to severe cellular malfunction, resulting in cell death or uncontrolled growth and malignancy. Indeed, the MYST family HATs have been implicated in several forms of human cancer. (Avvakumov, N. et al. “The MYST family of histone acetyltransferases and their intimate links to cancer.” Oncogene 26.37 (2007): 5395-5407.)

MOZ (monocytic leukaemia zinc finger protein) is known as oncogene in human. MOZ plays a key role as transcriptional coactivator and epigenetic regulator in the process of proliferation and the differentiation of hematopoietic progenitor and stem cells. The insights into the deregulation of these processes indicate that MOZ fusion proteins are related to the formation of leukemic stem cells and interfere with the activities of key proteins such as transcription factors, which render MOZ as a promising target for acute myeloid leukaemia therapy. Targeting MOZ by small molecules will hold promise for acute myeloid leukaemia therapy. (Zhou C. et al. “MOZ/KAT6A: a promising target for acute myeloid leukaemia therapy.” (2020): 759-761)

Cellular senescence plays a key role to restrict tumour growth. KAT6A represses cellular senescence in mouse embryonic fibroblasts (MEFs) while not affecting apoptosis or DNA damage. MOZ directly binds to genes that inhibit senescence including Cdc6, E2f2, Ezh2 and Melk, and in its absence, H3K9ac and H3K27ac at the TSS of these loci is reduced. (Sheikh, B. N., et al. “MOZ (MYST3, KAT6A) inhibits senescence via the INK4A-ARF pathway.” Oncogene 34.47 (2015): 5807-5820)

Histone acetyltransferase KAT6A-upregulated PJ3K/AKT signalling through TRIM24 binding is critical for cell proliferation and tumour growth in gliomas. KAT6A promotes H3K23 acetylation and association with TRIM24, leading to increased PIK3CA expression and PI3K/Akt signalling activation, resulting in enhanced glioma tumorigenesis. KAT6A functions as an oncogene in gliomas. (Lv, D., et al. “Histone acetyltransferase KAT6A upregulates PJ3K/AKT signalling through TRIM24 binding.” Cancer research 77.22 (2017): 6190-6201).

Many patent publications disclose several small molecule compounds and their derivatives capable of targeting KAT target proteins. Considering the established role of KATs in diseases such as cancer and also because there are currently no FDA-approved targeted therapeutics for specific KAT6A or KAT6B target protein, there is a need for the development of compounds, compositions, and methods for treating KAT6A- or KAT6B-activated proliferative disorders and autoimmune diseases.

### SUMMARY OF THE INVENTION

Provided herein are compounds represented by compound of formula (I) or a pharmaceutically acceptable salt or a stereoisomer or a tautomer thereof and pharmaceutical compositions comprising compound of formula (I) or a pharmaceutically acceptable salt or a stereoisomer or a tautomer thereof that are useful as KAT6A inhibitors and for the treatment of diseases or disorders dependent upon or mediated by KAT6A. The present invention also provides a preparation of compound of formula (I) or a pharmaceutically acceptable salt or a stereoisomer or a tautomer thereof.

In one aspect, the present invention provides a compound of formula (I):

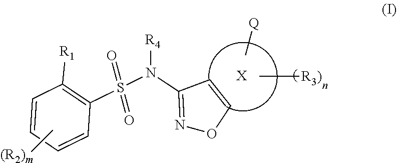

or a pharmaceutically acceptable salt or a stereoisomer or a tautomer thereof; wherein,
R₁ represents hydrogen, halogen, alkyl, alkoxy, haloalkoxy or aryl; wherein the alkyl is unsubstituted or substituted with one or more substituents selected from hydroxy, halogen, amino and amido; and the aryl is unsubstituted or substituted with one or more substituents selected from alkyl, alkoxy, hydroxy, cyano, halogen and amino;
R₂, at each occurrence, represents hydrogen, halogen, alkyl or alkoxy; wherein the alkyl and alkoxy are unsubstituted or substituted with one or more substituents selected from hydroxy, halogen, amino and amido;
X represents 6-membered fused heteroaryl ring or 6-membered fused heterocycloalkyl ring;
R₃, at each occurrence, represents hydrogen, alkyl, alkoxy or heteroaralkyl; wherein the alkyl and alkoxy groups are unsubstituted or substituted with one or more substituents selected from hydroxy, amino, halogen and amido; or
any two R₃ groups attached to the same carbon atom combine together to form an oxo group;
R₄ represents hydrogen or alkyl; wherein the alkyl is unsubstituted or substituted with one or more hydroxy or halogen groups;
Q represents hydrogen, —NRₐR_{b}, alkyl, aryl, 4- to 6-membered heterocycloalkyl, 3- to 8-membered cycloalkenyl or 5- to 6-membered heteroaryl; wherein the aryl, heterocycloalkyl, cycloalkenyl and heteroaryl are unsubstituted or substituted with one or more substituents selected from alkyl, alkoxy, amino, amido, halogen, haloalkyl, hydroxyalkyl and oxo; and the alkyl is unsubstituted or substituted with 5- to 6-membered heteroaryl;
Rₐ and R_{b} are independently selected from hydrogen, alkyl, 5- to 6-membered cycloalkyl, aryl and aryl-alkyl; wherein the cycloalkyl, aryl, and aryl-alkyl are unsubstituted or substituted with one or more substituents selected from alkyl, alkoxy, amino, halogen, haloalkyl and hydroxyalkyl;
m represents 1, 2 or 3; and
n represents 1, 2 or 3.

In yet another aspect, the present invention provides a pharmaceutical composition comprising a compound of formula (I) or a pharmaceutically acceptable salt or a stereoisomer or a tautomer thereof and at least one pharmaceutically acceptable excipient (such as a pharmaceutically acceptable carrier or diluent).

In another aspect, the present invention provides a pharmaceutical composition comprising a compound of formula (I) or a pharmaceutically acceptable salt or a stereoisomer or a tautomer thereof for use as a medicament.

In another aspect, the present invention provides a pharmaceutical composition comprising a compound of formula (I) or a pharmaceutically acceptable salt or a stereoisomer or a tautomer thereof for treating diseases or disorders that are dependent upon or mediated by KAT6A.

In another aspect, the present invention provides a pharmaceutical composition comprising a compound of formula (I) or a pharmaceutically acceptable salt or a stereoisomer or a tautomer thereof for treating diseases or disorders mediated by alterations in KAT6A protein including mutations and overexpression thereof.

In another aspect, the present invention provides methods for treating a disease or a disorder comprising administering a therapeutically effective amount of a compound represented by compound of formula (I) or a pharmaceutically acceptable salt or a stereoisomer or a tautomer thereof to a subject, e.g., a human, in need thereof. The disease or disorder, for example, cancer is treatable by inhibition of KAT6A.

In another aspect, the present invention provides a use of a compound represented by compound of formula (I) or a pharmaceutically acceptable salt or a stereoisomer or a tautomer thereof for the manufacture of a medicament for treating a disease or a condition, e.g., cancer.

### DETAILED DESCRIPTION OF THE INVENTION

The present invention provides fused isoxazolyl derivatives, referred as a compound of formula (I), which are useful as KAT6A inhibitors and for the treatment of conditions dependent upon or mediated by KAT6A. The present invention further provides pharmaceutical compositions comprising the said compound or a stereoisomer or a tautomer thereof as therapeutic agents.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In one embodiment, the present invention provides a compound of formula (I)

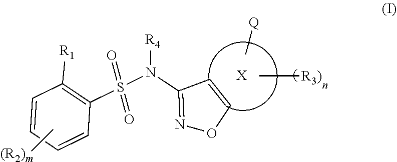

or a pharmaceutically acceptable salt or a stereoisomer or a tautomer thereof; wherein,
R₁ represents hydrogen, halogen, alkyl, alkoxy, haloalkoxy or aryl; wherein the alkyl is unsubstituted or substituted with one or more substituents selected from hydroxy, halogen, amino and amido; and the aryl is unsubstituted or substituted with one or more substituents selected from alkyl, alkoxy, hydroxy, cyano, halogen and amino;
R₂, at each occurrence, represents hydrogen, halogen, alkyl or alkoxy; wherein the alkyl and alkoxy are unsubstituted or substituted with one or more substituents selected from hydroxy, halogen, amino and amido;
X represents 6-membered fused heteroaryl ring or 6-membered fused heterocycloalkyl ring;
R₃, at each occurrence, represents hydrogen, alkyl, alkoxy or heteroaralkyl; wherein the alkyl and alkoxy groups are unsubstituted or substituted with one or more substituents selected from hydroxy, amino, halogen and amido; or
any two R₃ groups attached to the same carbon atom combine together to form an oxo group;
R₄ represents hydrogen or alkyl; wherein the alkyl is unsubstituted or substituted with one or more hydroxy or halogen groups;
Q represents hydrogen, —NRₐR_{b}, alkyl, aryl, 4- to 6-membered heterocycloalkyl, 3- to 8-membered cycloalkenyl or 5- to 6-membered heteroaryl; wherein the aryl, heterocycloalkyl, cycloalkenyl and heteroaryl are unsubstituted or substituted with one or more substituents selected from alkyl, alkoxy, amino, amido, halogen, haloalkyl, hydroxyalkyl and oxo; and the alkyl is unsubstituted or substituted with 5- to 6-membered heteroaryl;
Rₐ and R_{b} are independently selected from hydrogen, alkyl, 5- to 6-membered cycloalkyl, aryl and aryl-alkyl; wherein the cycloalkyl, aryl, and aryl-alkyl are unsubstituted or substituted with one or more substituents selected from alkyl, alkoxy, amino, halogen, haloalkyl and hydroxyalkyl;
m represents 1, 2 or 3; and
n represents 1, 2 or 3.

In one embodiment, R₁ represents hydrogen, halogen, (C₁-C₄)alkyl, (C₁-C₄)alkoxy, halo(C₁-C₄)alkoxy or aryl.

In one embodiment, R₁ represents hydrogen or (C₁-C₄)alkoxy.

In one embodiment, R₂, at each occurrence, represents hydrogen, halogen, (C₁-C₄)alkyl or (C₁-C₄)alkoxy.

In one embodiment, R₂, at each occurrence, represents hydrogen or (C₁-C₄)alkoxy.

In one embodiment, X represents 6-membered fused heteroaryl ring.

In one embodiment, X represents 6-membered fused heteroaryl ring containing 1, 2 or 3 N atoms.

In one embodiment, X represents

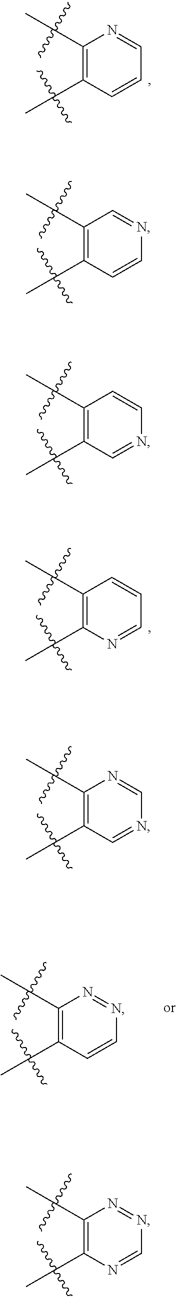

wherein [symbol] represents the points of fusion with isoxazolyl ring of formula (I).

In one embodiment, X represents

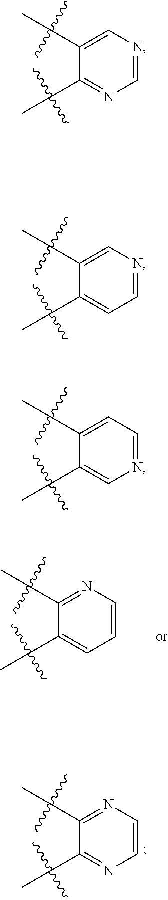

wherein [symbol] represents point of fusion with isoxazolyl ring of formula (I).

In one embodiment, the formula

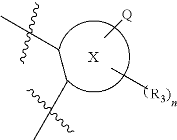

represents

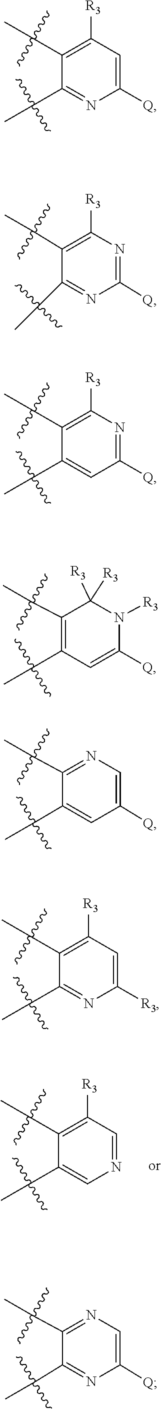

wherein [symbol] represents point of fusion with isoxazolyl ring of formula (I).

In one embodiment, R₃, at each occurrence, represents hydrogen, (C₁-C₄)alkyl, or (C₁-C₄)alkoxy.

In one embodiment, any two R₃ groups attached to the same carbon atom combine together to form an oxo group.

In one embodiment, Q represents hydrogen, —NRₐR_{b}, phenyl, 4- to 6-membered heterocycloalkyl, 3- to 8-membered cycloalkenyl, 5- to 6-membered heteroaryl or an alkyl group substituted with 5- to 6-membered heteroaryl.

In one embodiment, Rₐ and R_{b} are independently selected from hydrogen, (C₁-C₄)alkyl, 5- to 6-membered cycloalkyl, aryl and aryl-alkyl.

In one embodiment, Q represents,
(i) hydrogen, —NH(CH₃), —N(CH₃)(CH₃), —N(CH₂CH₃)(CH₂CH₃), —NH(CH₂CH₃), —N(CH₃)(CH₂CH₃), —N(CH₃)(phenyl), —N(CH₃)(cyclohexyl), —N(CH₂CH₃)(phenyl), —N(CH₂CH₃)(CH₂-phenyl), —N(CH₃)(CH₂-phenyl), —N(CH₂CH₃)(cyclohexyl) or —CH₂-pyrazolyl; wherein the phenyl, cyclohexyl, and pyrazolyl are unsubstituted or substituted with 1 or 2 substituents selected from alkyl, alkoxy, halogen, haloalkyl and hydroxyalkyl; or
(ii) phenyl, azetidinyl, thietanyl, oxetanyl, oxazetidinyl, pyrrolidinyl, tetrahydrofuranyl, tetrahydrothiophenyl, imidazolidinyl, oxazolidinyl, thiazolidinyl, piperidinyl, tetrahydropyranyl, morpholinyl, dioxanyl, cyclopentenyl, cyclohexenyl, furanyl, thiophenyl, pyrrolyl, oxazolyl, imdazolyl, thiazolyl, isothiazolyl, pyrazolyl, isoxazolyl, pyridinyl, pyranyl, pyridazinyl, pyrimidinyl or pyrazinyl; wherein each group is unsubstituted or substituted with 1 or 2 substituents selected from alkyl, alkoxy, halogen, haloalkyl and hydroxyalkyl.

In one embodiment, R₄ represents hydrogen or (C₁-C₄)alkyl.

In one embodiment, the present invention provides compound of formula (I), wherein
R₁ represents hydrogen, halogen, (C₁-C₄)alkyl, (C₁-C₄)alkoxy, halo(C₁-C₄)alkoxy or aryl;
R₂, at each occurrence, represents hydrogen, halogen, (C₁-C₄)alkyl or (C₁-C₄)alkoxy;
X represents

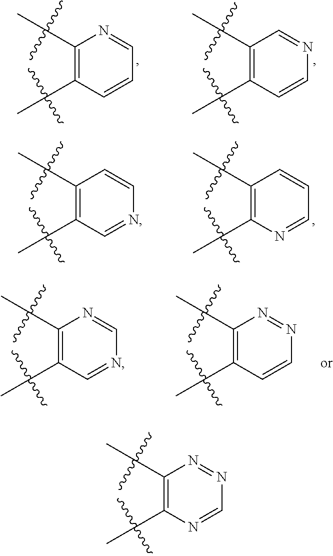

wherein [symbol] represents the points of fusion with isoxazolyl ring of formula (I);
R₃, at each occurrence, represents hydrogen, (C₁-C₄)alkyl or (C₁-C₄)alkoxy; or any two R₃ groups attached to the same carbon atom combine together to form an oxo group; and
   Q represents,
(i) hydrogen, —NH(CH₃), —N(CH₃)(CH₃), —N(CH₂CH₃)(CH₂CH₃), —NH(CH₂CH₃), —N(CH₃)(CH₂CH₃), —N(CH₃)(phenyl), —N(CH₃)(cyclohexyl), —N(CH₂CH₃)(phenyl), —N(CH₂CH₃)(CH₂-phenyl), —N(CH₃)(CH₂-phenyl), —N(CH₂CH₃)(cyclohexyl) or —CH₂-pyrazolyl; wherein the phenyl, cyclohexyl, and pyrazolyl are unsubstituted or substituted with 1 or 2 substituents selected from alkyl, alkoxy, halogen, haloalkyl and hydroxyalkyl; or
(ii) phenyl, azetidinyl, thietanyl, oxetanyl, oxazetidinyl, pyrrolidinyl, tetrahydrofuranyl, tetrahydrothiophenyl, imidazolidinyl, oxazolidinyl, thiazolidinyl, piperidinyl, tetrahydropyranyl, morpholinyl, dioxanyl, cyclopentenyl, cyclohexenyl, furanyl, thiophenyl, pyrrolyl, oxazolyl, imdazolyl, thiazolyl, isothiazolyl, pyrazolyl, isoxazolyl, pyridinyl, pyranyl, pyridazinyl, pyrimidinyl or pyrazinyl; wherein each group is unsubstituted or substituted with 1 or 2 substituents selected from alkyl, alkoxy, halogen, haloalkyl and hydroxyalkyl.

In one embodiment, the present invention provides compound of formula (IA) or a pharmaceutically acceptable salt or a stereoisomer or a tautomer thereof:

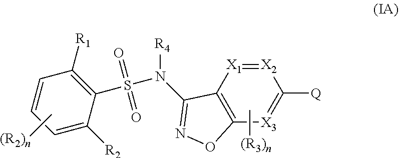

wherein,
[symbol] represents a single bond or double bond;
X₁, X₂ and X₃ independently represents N or C; wherein at least one of X₁, X₂ and X₃ is N; and
R₁, R₂, R₃, R₄, Q, m and n are as defined in compound of formula (I).

In one embodiment, the present invention provides a compound of formula (IA),
R₁ represents hydrogen, halogen, (C₁-C₄) alkyl, (C₁-C₄)alkoxy, halo-(C₁-C₄)alkoxy or aryl;
R₂, at each occurrence, represents hydrogen, halogen, (C₁-C₄)alkyl or (C₁-C₄)alkoxy;
R₃, at each occurrence, represents hydrogen, (C₁-C₄)alkyl or (C₁-C₄)alkoxy; or any two R₃ groups attached to the same carbon atom combine together to form an oxo group;
R₄ represents hydrogen or (C₁-C₄)alkyl; and
Q represents,
(i) hydrogen, —NH(CH₃), —N(CH₃)(CH₃), —NH(CH₂CH₃), —N(CH₃)(phenyl), —N(CH₃)(cyclohexyl), —N(CH₂CH₃)(phenyl), —N(CH₂CH₃)(CH₂-phenyl), —N(CH₃)(CH₂-phenyl), —N(CH₂CH₃)(cyclohexyl) or —CH₂-pyrazolyl; wherein the phenyl, cyclohexyl, and pyrazolyl are unsubstituted or substituted with 1 or 2 substituents selected from alkyl, alkoxy, halogen, haloalkyl and hydroxyalkyl; or
(ii) phenyl, azetidinyl, oxetanyl, pyrrolidinyl, tetrahydrofuranyl, imidazolidinyl, oxazolidinyl, thiazolidinyl, piperidinyl, tetrahydropyranyl, morpholinyl, cyclopentenyl, cyclohexenyl, furanyl, thiophenyl, pyrrolyl, isothiazolyl, pyrazolyl, isoxazolyl, pyridinyl, pyranyl, pyridazinyl, pyrimidinyl or pyrazinyl; wherein each group is unsubstituted or substituted with 1 or 2 substituents selected from alkyl, alkoxy, halogen and haloalkyl;
m represents 1 or 2; and
n represents 1, 2 or 3.

In one embodiment of formula (IA), [symbol] represents a single bond or double bond.

In one embodiment of compound of formula (IA), X₁, X₂ and X₃ independently represents N or C; wherein at least one of X₁, X₂ and X₃ is N.

In one embodiment of compound of formula (IA), R₄ represents hydrogen or (C₁-C₄)alkyl.

In one embodiment, the present invention provides compound of formula (IA), wherein
R₁ represents hydrogen, halogen, (C₁-C₄) alkyl, (C₁-C₄)alkoxy, halo-(C₁-C₄)alkoxy or aryl;
R₂, at each occurrence, represents hydrogen, halogen, (C₁-C₄)alkyl or (C₁-C₄)alkoxy;
R₃, at each occurrence, represents hydrogen, (C₁-C₄)alkyl or (C₁-C₄)alkoxy; or any two R₃ groups attached to the same carbon atom combine together to form an oxo group;
R₄ represents hydrogen or (C₁-C₄)alkyl; and
Q represents,
(i) hydrogen, —NH(CH₃), —N(CH₃)(CH₃), —NH(CH₂CH₃), —N(CH₃)(phenyl), —N(CH₃)(cyclohexyl), —N(CH₂CH₃)(phenyl), —N(CH₂CH₃)(CH₂-phenyl), —N(CH₃)(CH₂-phenyl), —N(CH₂CH₃)(cyclohexyl) or —CH₂-pyrazolyl; wherein the phenyl, cyclohexyl, and pyrazolyl are unsubstituted or substituted with 1 or 2 substituents selected from alkyl, alkoxy, halogen, haloalkyl and hydroxyalkyl; or
(ii) phenyl, azetidinyl, oxetanyl, pyrrolidinyl, tetrahydrofuranyl, imidazolidinyl, oxazolidinyl, thiazolidinyl, piperidinyl, tetrahydropyranyl, morpholinyl, cyclopentenyl, cyclohexenyl, furanyl, thiophenyl, pyrrolyl, isothiazolyl, pyrazolyl, isoxazolyl, pyridinyl, pyranyl, pyridazinyl, pyrimidinyl or pyrazinyl; wherein each group is unsubstituted or substituted with 1 or 2 substituents selected from alkyl, alkoxy, halogen and haloalkyl;
m represents 1 or 2; and
n represents 1, 2 or 3.

In one embodiment, the present invention provides a compound of formula (IB) or a pharmaceutically acceptable salt or a stereoisomer or a tautomer thereof:

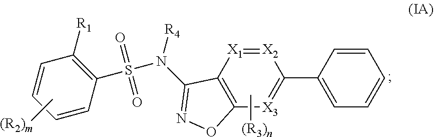

wherein,
X₁, X₂ and X₃ independently represents N or C; wherein at least one of X₁, X₂ and X₃ is N; and
R₁, R₂, R₃, R₄, m and n are as defined in compound of formula (I).

In one embodiment, the present invention provides a compound of formula (IB), wherein:
R₁ represents hydrogen, —Cl, —F, —O—CH₃, —O—CH₂CH₃, —O—CF₃ or phenyl;
R₂, at each occurrence, represents —Cl, —F, —CH₃, —CH₂CH₃, —C(CH₃)₃, —O—CH₃ or —O—CH₂CH₃;
R₃, at each occurrence, represents hydrogen, —CH₃, —CH₂CH₃, —C(CH₃)₃, —O—CH₃, —O—CH₂CH₃; or any two R₃ groups attached to the same carbon atom combine together to form an oxo group;
m represents 1 or 2; and
n represents 1, 2 or 3.

In one embodiment, the present invention provides compound of formula (IC) or a pharmaceutically acceptable salt or a stereoisomer or a tautomer thereof:

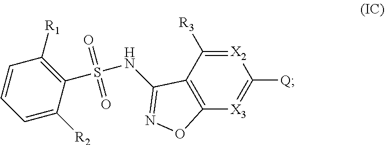

wherein,
X₂ and X₃ independently represents N or C; wherein at least one of X₂ and X₃ is N;
Q represents 4- to 6-membered heterocycloalkyl, 3- to 8-membered cycloalkenyl or 5- to 6-membered heteroaryl; and
R₁, R₂, R₃, Q, m and n are as defined in compound of formula (I).

In one embodiment of compound of formula (IC), Q represents

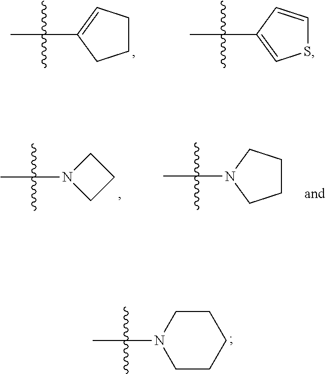 [SMILES: *C1=CCCC1 ; *c1ccsc1 ; *N1CCC1 ; *N1CCCC1 ; *N1CCCCC1]

wherein each group is unsubstituted or substituted with one or two occurrences of —Cl, —F, —OCH₃ or —OCH₂CH₃.

In one embodiment, the present invention provides a compound of formula (IC), R₁ represents hydrogen, —Cl, —F, —O—CH₃, —O—CH₂CH₃, —O—CF₃ or phenyl;
R₂, at each occurrence, represents hydrogen, —Cl, —F, —CH₃, —CH₂CH₃, —C(CH₃)₃, —O—CH₃ or —O—CH₂CH₃;
R₃, at each occurrence, represents hydrogen, —CH₃, —CH₂CH₃, —C(CH₃)₃, —O—CH₃, —O—CH₂CH₃; or any two R₃ groups attached to the same carbon atom to form an oxo group; and
Q represents

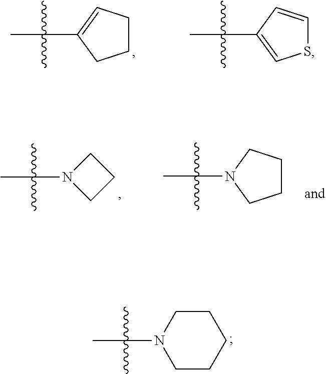 [SMILES: *C1=CCCC1 ; *c1ccsc1 ; *N1CCC1 ; *N1CCCC1 ; *N1CCCCC1]

wherein each group is unsubstituted or substituted with one or two occurrences of —Cl, —F, —OCH₃ or —OCH₂CH₃.

In one embodiment, the present invention provides a compound selected from:

| Compound No. | Structure |
|---|---|
| 1 | 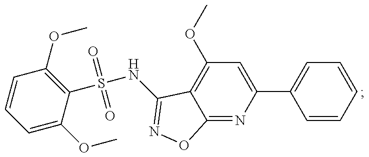 [SMILES: COc1cccc(OC)c1S(=O)(=O)Nc1noc2nc(-c3ccccc3)cc(OC)c12] |
| 2 | 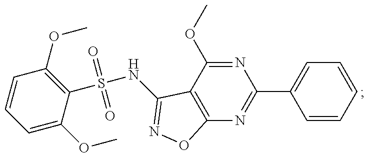 [SMILES: COc1cccc(OC)c1S(=O)(=O)Nc1noc2nc(-c3ccccc3)nc(OC)c12] |
| 3 | 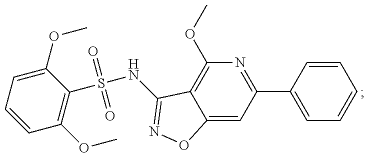 [SMILES: COc1cccc(OC)c1S(=O)(=O)Nc1noc2cc(-c3ccccc3)nc(OC)c12] |
| 4 | 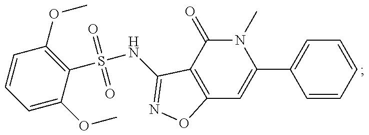 [SMILES: COc1cccc(OC)c1S(=O)(=O)Nc1noc2cc(-c3ccccc3)n(C)c(=O)c12] |
| 5 | 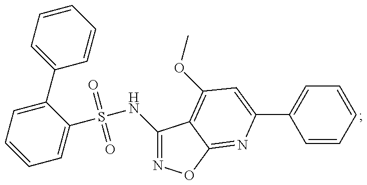 [SMILES: COc1cc(-c2ccccc2)nc2onc(NS(=O)(=O)c3ccccc3-c3ccccc3)c12] |
| 6 | 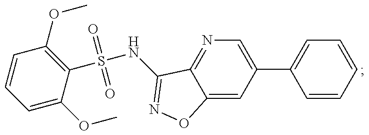 [SMILES: COc1cccc(OC)c1S(=O)(=O)Nc1noc2cc(-c3ccccc3)cnc12] |
| 7 | 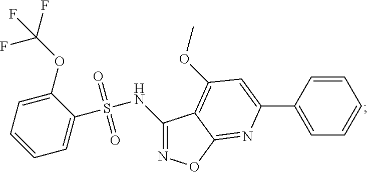 [SMILES: COc1cc(-c2ccccc2)nc2onc(NS(=O)(=O)c3ccccc3OC(F)(F)F)c12] |
| 8 | 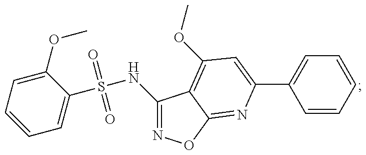 [SMILES: COc1ccccc1S(=O)(=O)Nc1noc2nc(-c3ccccc3)cc(OC)c12] |
| 9 | 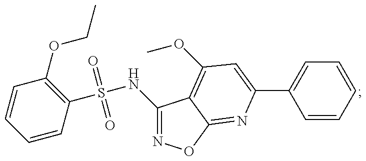 [SMILES: CCOc1ccccc1S(=O)(=O)Nc1noc2nc(-c3ccccc3)cc(OC)c12] |
| 10 | 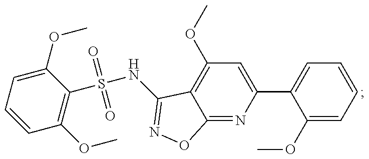 [SMILES: COc1ccccc1-c1cc(OC)c2c(NS(=O)(=O)c3c(OC)cccc3OC)noc2n1] |
| 11 | 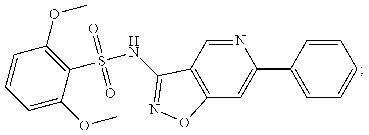 [SMILES: COc1cccc(OC)c1S(=O)(=O)Nc1noc2cc(-c3ccccc3)ncc12] |
| 12 | 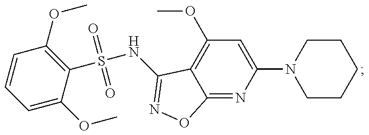 [SMILES: COc1cccc(OC)c1S(=O)(=O)Nc1noc2nc(N3CCCCC3)cc(OC)c12] |
| 13 | 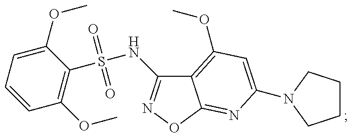 [SMILES: COc1cccc(OC)c1S(=O)(=O)Nc1noc2nc(N3CCCC3)cc(OC)c12] |
| 14 | 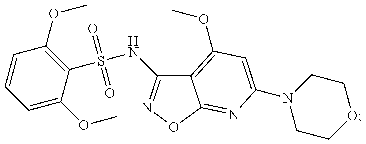 [SMILES: COc1cccc(OC)c1S(=O)(=O)Nc1noc2nc(N3CCOCC3)cc(OC)c12] |
| 15 | 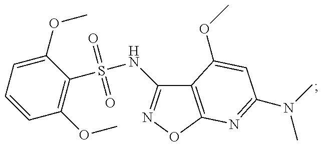 [SMILES: COc1cccc(OC)c1S(=O)(=O)Nc1noc2nc(N(C)C)cc(OC)c12] |
| 16 | 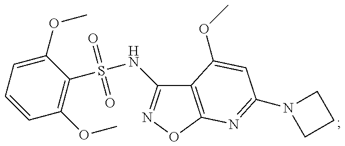 [SMILES: COc1cccc(OC)c1S(=O)(=O)Nc1noc2nc(N3CCC3)cc(OC)c12] |
| 17 | 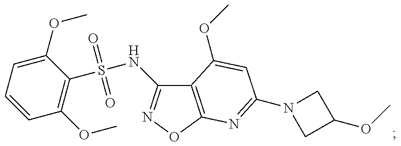 [SMILES: COc1cccc(OC)c1S(=O)(=O)Nc1noc2nc(N3CC(OC)C3)cc(OC)c12] |
| 18 | 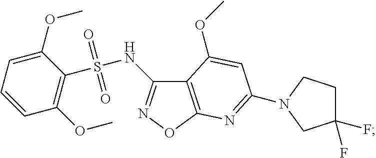 [SMILES: COc1cccc(OC)c1S(=O)(=O)Nc1noc2nc(N3CCC(F)(F)C3)cc(OC)c12] |
| 19 | 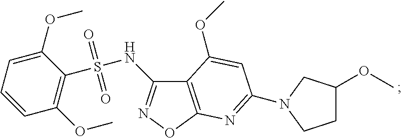 [SMILES: COc1cccc(OC)c1S(=O)(=O)Nc1noc2nc(N3CCC(OC)C3)cc(OC)c12] |
| 20 | 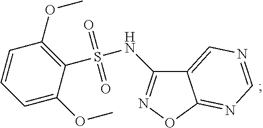 [SMILES: COc1cccc(OC)c1S(=O)(=O)Nc1noc2ncncc12] |
| 21 | 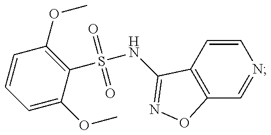 [SMILES: COc1cccc(OC)c1S(=O)(=O)Nc1noc2cnccc12] |
| 22 | 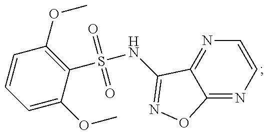 [SMILES: COc1cccc(OC)c1S(=O)(=O)Nc1noc2nccnc12] |
| 23 | 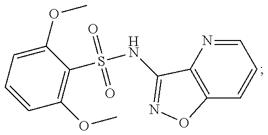 [SMILES: COc1cccc(OC)c1S(=O)(=O)Nc1noc2cccnc12] |
| 24 | 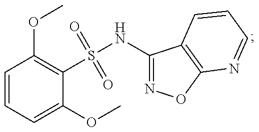 [SMILES: COc1cccc(OC)c1S(=O)(=O)Nc1noc2ncccc12] |
| 25 | 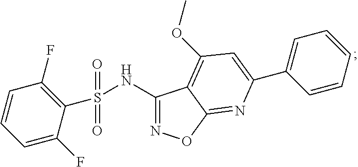 [SMILES: COc1cc(-c2ccccc2)nc2onc(NS(=O)(=O)c3c(F)cccc3F)c12] |
| 26 | 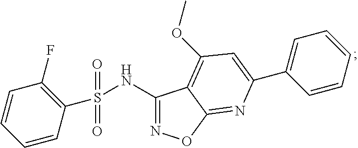 [SMILES: COc1cc(-c2ccccc2)nc2onc(NS(=O)(=O)c3ccccc3F)c12] |
| 27 | 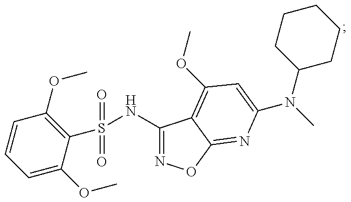 [SMILES: COc1cccc(OC)c1S(=O)(=O)Nc1noc2nc(N(C)C3CCCCC3)cc(OC)c12] |
| 28 | 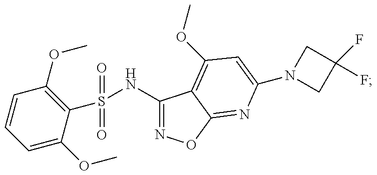 [SMILES: COc1cccc(OC)c1S(=O)(=O)Nc1noc2nc(N3CC(F)(F)C3)cc(OC)c12] |
| 29 | 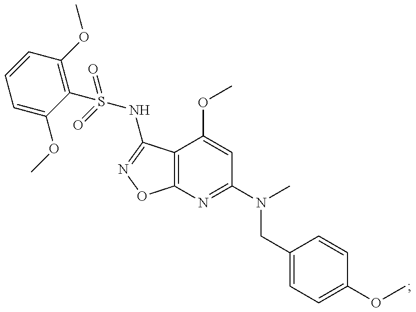 [SMILES: COc1ccc(CN(C)c2cc(OC)c3c(NS(=O)(=O)c4c(OC)cccc4OC)noc3n2)cc1] |
| 30 | 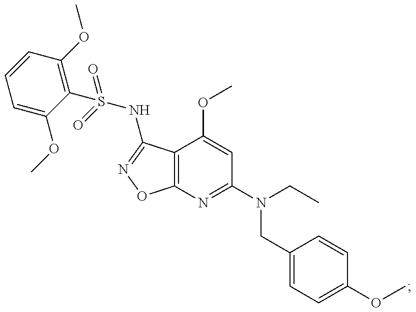 [SMILES: CCN(Cc1ccc(OC)cc1)c1cc(OC)c2c(NS(=O)(=O)c3c(OC)cccc3OC)noc2n1] |
| 31 | 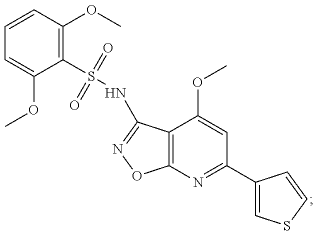 [SMILES: COc1cccc(OC)c1S(=O)(=O)Nc1noc2nc(-c3ccsc3)cc(OC)c12] |
| 32 | 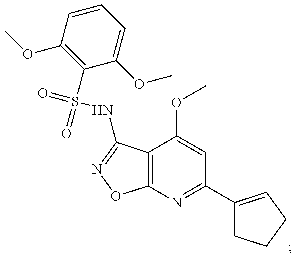 [SMILES: COc1cccc(OC)c1S(=O)(=O)Nc1noc2nc(C3=CCCC3)cc(OC)c12] |
| 33 | 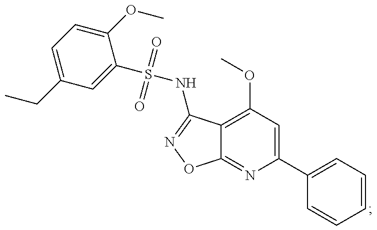 [SMILES: CCc1ccc(OC)c(S(=O)(=O)Nc2noc3nc(-c4ccccc4)cc(OC)c23)c1] |
| 34 | 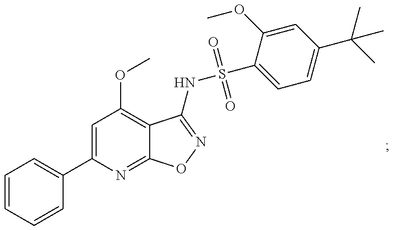 [SMILES: COc1cc(C(C)(C)C)ccc1S(=O)(=O)Nc1noc2nc(-c3ccccc3)cc(OC)c12] |
| 35 | 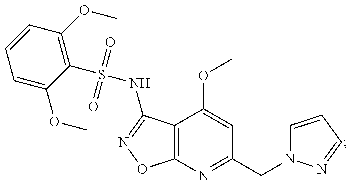 [SMILES: COc1cccc(OC)c1S(=O)(=O)Nc1noc2nc(Cn3cccn3)cc(OC)c12] |
| 36 | 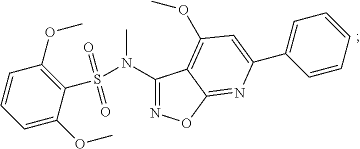 [SMILES: COc1cccc(OC)c1S(=O)(=O)N(C)c1noc2nc(-c3ccccc3)cc(OC)c12] |
| 37 | 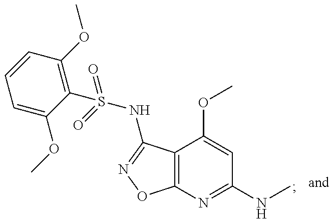 [SMILES: CNc1cc(OC)c2c(NS(=O)(=O)c3c(OC)cccc3OC)noc2n1] |
| 38 | 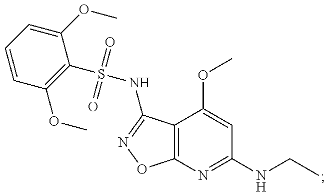 [SMILES: CCNc1cc(OC)c2c(NS(=O)(=O)c3c(OC)cccc3OC)noc2n1] |

or a pharmaceutically acceptable salt or a stereoisomer or a tautomer thereof.

### Method of Treatment

In one embodiment, the present invention provides a compound of formula (I) or a pharmaceutical acceptable salt or a stereoisomer or a tautomer thereof, for use in the treatment of diseases or disorders dependent upon KAT6A.

In one embodiment, the present invention provides a pharmaceutical composition comprising compound of formula (I) or a pharmaceutical acceptable salt or a stereoisomer or a tautomer thereof, for use in the treatment of diseases or disorders dependent upon KAT6A.

In one embodiment, the present invention provides a method of inhibiting KAT6A comprising administering to a cell, in need thereof, a therapeutically effective amount of a compound of formula (I) or a pharmaceutically acceptable salt or a stereoisomer or a tautomer thereof.

In one embodiment, the present invention provides a method of modulating KAT6A in a subject comprising administering to the subject, in need thereof, a therapeutically effective amount of a compound of formula (I) or a pharmaceutically acceptable salt thereof or a stereoisomer or a tautomer thereof.

In one embodiment, the present invention provides a method for treating a disease or disorder mediated by KAT6A, in a subject comprising administering to the subject, in need thereof, a therapeutically effective amount of a compound of formula (I) or a pharmaceutically acceptable salt thereof or a stereoisomer or a tautomer thereof.

In one embodiment, the present invention provides a pharmaceutical composition comprising a compound of formula (I) or a pharmaceutically acceptable salt thereof or a stereoisomer or a tautomer thereof as described herein and at least one pharmaceutically acceptable excipient (such as a pharmaceutically acceptable carrier or diluent). Preferably, the pharmaceutical composition comprises a therapeutically effective amount of at least one compound described herein. The compounds described in the present invention may be associated with a pharmaceutically acceptable excipient (such as a carrier or a diluent) or be diluted by a carrier or enclosed within a carrier which can be in the form of a capsule, sachet, paper or other container.

In one embodiment, the present invention provides a pharmaceutical composition comprising a compound of formula (I), for use in the manufacture of a medicament.

In one embodiment, the present invention provides a pharmaceutical composition comprising a compound of formula (I), for use in in the treatment of a disease or disorder mediated by the inhibition of KAT6A. In one embodiment, the disease or disorder is a cancer.

In one embodiment, the cancer is selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer or virus-related cancer.

In one embodiment, the present invention provides a compound or a pharmaceutically acceptable salt or a stereoisomer or a tautomer thereof, for use as a medicament.

In one embodiment, the present invention provides a use of pharmaceutical composition comprising a compound represented by formula (I), in the manufacture of a medicament for treating a disease or disorder mediated by KAT6A.

In one embodiment, the present invention provides a use of compound represented by the formula (I) or a pharmaceutically acceptable salt or a stereoisomer or a tautomer thereof, in the manufacture of a medicament for treating or preventing a disease or disorder mediated by KAT6A.

In one embodiment, a disease or disorder dependent upon or mediated by KAT6A, includes cancer.

In one embodiment, cancer is selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer or virus-related cancer.

In one embodiment, the present invention provides a use of compound represented by formula (I) or a pharmaceutically acceptable salt or a stereoisomer or a tautomer thereof, in the manufacture of a medicament for the treatment of cancer selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductaladenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer or virus-related cancer.

In certain embodiments, the present invention provides a compound represented by formula (I), or a pharmaceutically acceptable salt or a stereoisomer or a tautomer thereof, for use in the treatment of a disease or disorder mediated by KAT6A.

In one embodiment, the present invention provides a compound of formula (I), or a pharmaceutically acceptable salt or a stereoisomer or a tautomer thereof, for use in treating or preventing cancer selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer or virus-related cancer.

### Pharmaceutical Compositions

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions can also include adjuvants such as wetting agents, emulsifying and suspending agents, sweetening, flavoring and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, including but not limited to tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to the compounds of this application, excipients such as lactose, talc, silicic acid, aluminum hydroxide, calcium silicates and polyamide powder or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Administration of the disclosed compounds and pharmaceutical compositions can be accomplished via any mode of administration for therapeutic agents. These modes include systemic or local administration such as oral, nasal, parenteral, intravenous, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions or the like, sometimes in unit dosages and consistent with conventional pharmaceutical practices. Likewise, they can also be administered in intravenous (both bolus and infusion), intraperitoneal, subcutaneous or intramuscular form and all using forms well known to those skilled in the pharmaceutical arts.

Illustrative pharmaceutical compositions are tablets and gelatin capsules comprising one or more compounds of the present disclosure and a pharmaceutically acceptable carrier, such as, but not limited to, a) a diluent, e.g., purified water, triglyceride oils, such as hydrogenated or partially hydrogenated vegetable oil or mixtures thereof, corn oil, olive oil, sunflower oil, safflower oil, fish oils, such as EPA or DHA or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, one or more disclosed compound is dissolved in or mixed with a pharmaceutically acceptable solvent such as, for example, water, saline, aqueous dextrose, glycerol, ethanol and the like, to thereby form an injectable isotonic solution or suspension. Proteins such as albumin, chylomicron particles or serum proteins can be used to solubilize the disclosed compounds.

One or more disclosed compounds or compositions can be delivered by parental administration. The parental injectable administration is generally used for subcutaneous, intramuscular or intravenous injections and infusions. Injectables can be prepared in conventional forms, either as liquid solutions or suspensions or solid forms suitable for dissolving in liquid prior to injection.

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the terms “optional” or “optionally” mean that the subsequently described event or circumstance may occur or may not occur and that the description includes instances where the event or circumstance occurs as well as instances in which it does not. For example, “optionally substituted alkyl” refers to an event or circumstance in which the said alkyl may be substituted as well as the event or circumstance in which the alkyl is not substituted. The term “optionally substituted alkyl” can also be referred to ‘unsubstituted or substituted alkyl’ group.

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Unless specifically stated, the substituents can include any substituents described herein, for example, a halogen, a hydroxyl, a carbonyl (such as a carboxyl, an alkoxycarbonyl, a formyl or an acyl), a thiocarbonyl (such as a thioester, a thioacetate or a thioformate), an alkoxyl, an oxo, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heteroaryl, a heterocycloalkyl, an aralkyl or an aromatic or heteroaromatic moiety. It will be understood by those skilled in the art that substituents can themselves be substituted, if appropriate.

As used herein, the term “alkyl” refers to saturated aliphatic groups, including but not limited to C₁-C₁₀ straight-chain alkyl groups or C₃-C₁₀ branched-chain alkyl groups. Preferably, the “alkyl” group refers to C₁-C₄ straight-chain alkyl groups or C₃-C₆ branched-chain alkyl groups. Most preferably, the “alkyl” group refers to C₁-C₄ straight-chain alkyl groups or C₃-C₈ branched-chain alkyl groups. Examples of “alkyl” include, but are not limited to, methyl, ethyl, 1-propyl, 2-propyl, isopropyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl and 4-octyl. The “alkyl” group may be optionally substituted.

As used herein, the term ‘aryl-alkyl’ refers to a group wherein the ‘alkyl’ group is substituted with one or more ‘aryl’ groups.

As used herein, the term “halo” or “halogen” alone or in combination with other term(s) means fluorine, chlorine, bromine or iodine.

As used herein, the term “haloalkyl” refers to alkyl substituted with one or more halogen atoms, wherein the halo and alkyl groups are as defined above. Examples of “haloalkyl” include but are not limited to fluoromethyl, difluoromethyl, chloromethyl, trifluoromethyl and 2,2,2-trifluoroethyl.

As used herein, the term “hydroxyalkyl” refers to an alkyl group, as defined above, wherein one or more of the alkyl group's hydrogen atoms have been replaced with hydroxyl group. Examples of hydroxyalkyl moieties include but are not limited to —CH₂OH, —CH₂CH₂OH, —CH₂CH₂CH₂OH, —CH₂CH(OH)CH₂OH, —CH₂CH(OH) CH₃, —CH(CH₃)CH₂OH.

As used herein, the term “aryl”, as employed herein as such or as part of another group, refers to a monocyclic, bicyclic or polycyclic aromatic hydrocarbon ring system of 6 to 14 carbon atoms. Examples of aryl groups include, but are not limited to phenyl, naphthyl, biphenyl, anthryl and acenaphthyl. Preferred aryl group is phenyl.

As used herein, the term “cyano” refers to —CN group. As used herein, “amino” refers to an —NH₂ group. As used herein, “amido” refers to an —CONH₂ group.

As used herein, the term “cycloalkenyl” refers to a monocyclic or fused or bridged bicyclic carbocyclic ring system having one or more units of unsaturation but are not aromatic. For example, cycloalkenyl as used herein can be a C₃-C₁₀ monocyclic or fused or bridged C₈-C₁₂ bicyclic carbocyclic ring system having one, two or three units of unsaturation and are not aromatic. Preferred cycloalkenyl groups include but not limited to cyclopropenyl, cyclobutenyl, cyclopentenyl, cyclohexenyl, and cycloheptenyl.

As used herein, the term “cycloalkyl” alone or in combination with other term(s) means C₃-C₁₀ saturated cyclic hydrocarbon ring. A cycloalkyl may be a single ring, which typically contains from 3 to 7 carbon ring atoms. Examples of single-ring cycloalkyls include but are not limited to cyclopropyl, cyclobutyl, cyclopentyl, cyclohexyl and cycloheptyl. A cycloalkyl may alternatively be polycyclic or contain more than one ring. Examples of polycyclic cycloalkyls include bridged, fused and spirocyclic carbocyclyls.

As used herein, the term “heterocycloalkyl” refers to a non-aromatic, saturated or partially saturated, bridged bicyclic, monocyclic or polycyclic ring system of 3- to 15-member, unless the ring size is specifically mentioned, having at least one heteroatom selected from O, N or S with the remaining ring atoms being independently selected from the group consisting of carbon, oxygen, nitrogen and sulfur. The term “heterocycloalkyl” also refers to the bridged bicyclic ring system having at least one heteroatom selected from O, N or S. Examples of “heterocycloalkyl” include, but not limited to, azetidinyl, oxetanyl, imidazolidinyl, pyrrolidinyl, oxazolidinyl, thiazolidinyl, pyrazolidinyl, tetrahydrofuranyl, piperidinyl, dihydropyridinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, isoindolinyl, oxoisoindolinyl, dioxoisoindolinyl, aza-bicyclooctanyl, diazabicyclooctanyl, azocinyl, chromanyl, isochromanyl and xanthenyl. Attachment of a heterocycloalkyl substituent can occur via either a carbon atom or a heteroatom. A heterocycloalkyl group can be optionally substituted with one or more suitable groups by one or more aforesaid groups. Preferably “heterocycloalkyl” refers to 4- to 6-membered ring (unless the ring size is specifically mentioned) selected from the group consisting of azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl and thiomorpholinyl. All heterocycloalkyl are optionally substituted by one or more aforesaid groups.

As used herein, the term “heteroaryl” refers to a completely unsaturated ring system containing a total of 5 to 14 ring atoms, unless the ring size is specifically mentioned. At least one of the ring atoms is a heteroatom (i.e., 0, N or S), with the remaining ring atoms/groups being independently selected from C, N, O or S. A heteroaryl may be a single-ring (monocyclic) or multiple rings (bicyclic, tricyclic or polycyclic) fused together or linked covalently. Preferably, “heteroaryl” is a 5- to 6-membered ring, unless the ring size is specifically mentioned. The rings may contain from 1 to 4 additional heteroatoms selected from N, O and S, wherein the N atom is optionally quarternized. Any suitable ring position of the heteroaryl moiety may be covalently linked to the defined chemical structure. Examples of “heteroaryl” include but not limited to furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl (pyridinyl), 3-fluoropyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carbolinyl, indolizinyl, benzoisothiazolyl, benzoxazolyl, pyrrolopyridyl, purinyl, benzothiadiazolyl, benzooxadiazolyl, benzotriazolyl, benzotriadiazolyl, carbazolyl, dibenzothienyl, acridinyl and the like. Heteroaryl group may be optionally further substituted.

As used herein, “Fused” refers to a fusion of any heteroaryl ring or heterocycloalkyl ring which is subsequently described herein to the adjacent atoms of an existing ring structure in the compounds of the invention, wherein the groups heteroaryl and heterocycloalkyl are as defined above. In embodiment, such fusion is existing between the heteroaryl ring or heterocyclic ring with isoxazole ring of compound of formula (I). For instance, fused heteroaryl refers to any heteroaryl ring fused with isoxazole ring of compound of formula (I).

As used herein by themselves or in conjunction with another term or terms, “heteroaralkyl group” refers to an alkyl group in which a hydrogen atom is replaced by a heteroaryl group, wherein alkyl group and heteroaryl group are as previously defined. The heteroaralkyl groups can be substituted or unsubstituted.

As used herein, the term “amino” refers to an —NH₂ group.

As used herein, the term “hydroxy” or “hydroxyl” alone or in combination with other term(s) means —OH.

As used herein, the term “oxo” refers to ═O group.

As used herein, the term “alkoxy” refers to the group —O-alkyl, where the alkyl groups are as defined above. Exemplary C₁-C₁₀ alkoxy group include but are not limited to methoxy, ethoxy, n-propoxy, n-butoxy and t-butoxy. An alkoxy group can be optionally substituted with one or more suitable groups.

As used herein, the term “haloalkoxy” refers to an alkoxy group substituted with one or more halogen atoms (i.e., haloC₁-C₈alkoxy). Examples of “haloalkoxy” include but are not limited to fluoromethoxy, difluoromethoxy, trifluoromethoxy, 2,2,2-trifluoroethoxy, pentafluoroethoxy, pentachloroethoxy, chloromethoxy, dichlorormethoxy, trichloromethoxy and 1-bromoethoxy.

The term “heteroatom” as used herein designates a sulfur, nitrogen or oxygen atom.

As used herein, the term ‘compound(s)’ comprises the compound(s) disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or a pharmaceutically acceptable salt thereof; and a pharmaceutically acceptable carrier.

The pharmaceutical composition(s) usually contain(s) about 1% to 99%, for example, about 5% to 75% or from about 10% to about 30% by weight of the compound of formula (I) or (II) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the aforementioned range.

As used herein, “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant or emulsifier that has been approved by the United States Food and Drug Administration as being acceptable for use in humans or domestic animals.

The term “administer,” “administering,” or “administration” as used in this disclosure refers to either directly administering one or more disclosed compounds or a pharmaceutically acceptable salt of one or more disclosed compounds or a composition comprising one or more disclosed compounds to a subject or analog of the compound or a pharmaceutically acceptable salt of the compound or composition to the subject, which can form an equivalent amount of active compound within the subject's body.

The term “carrier” as used in this disclosure, encompasses carriers, excipients and diluents and means a material, composition or vehicle, such as a liquid or solid filler, diluent, excipient, solvent or encapsulating material, involved in carrying or transporting a pharmaceutical agent from one organ or portion of the body to another organ or portion of the body of a subject.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal and most preferably a human.

As used herein, the term, “therapeutically effective amount” refers to an amount of a compound of formula (I) or a pharmaceutically acceptable salt or a stereoisomer or a tautomer thereof; or a composition comprising the compound of formula (I) or a pharmaceutically acceptable salt or a stereoisomer or a tautomer thereof, effective in producing the desired therapeutic response in a particular patient suffering from a diseases or disorder, in particular their use in diseases or disorder associated with cancer. Particularly, the term “therapeutically effective amount” includes the amount of the compound of formula (I) or a pharmaceutically acceptable salt or a stereoisomer or a tautomer thereof, when administered, that induces a positive modification in the disease or disorder to be treated or is sufficient to prevent development of or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

The term, “pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

The term “pharmaceutically acceptable salt” refers to a product obtained by reaction of the compound of the present invention with a suitable acid or a base. In some cases, a medicament can be present in the form of a pharmaceutically acceptable salt. In some instances, a pharmaceutically acceptable salt can be a salt described in Berge et al, J. Pharm. Sci, 1977. In some instances, a pharmaceutically acceptable salts can include those salts derived from a mineral, organic acid or inorganic base. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids.

The pharmaceutically acceptable salts of the present invention can be prepared from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20^{th} ed., Mack Publishing Company, Easton, Pa., (1985); and in “Handbook of Pharmaceutical Salts: Properties, Selection and Use” by Stahl and Wermuth (Wiley-VCH, Weinheim, Germany, 2002).

The present invention also provides methods for formulating the disclosed compounds as for pharmaceutical administration.

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

The term “stereoisomers” refers to any enantiomers, diastereoisomers or geometrical isomers of the compound of formula (I), wherever they are chiral or when they bear one or more double bonds. When the compounds of the formula (I) and related formulae are chiral, they can exist in racemic or in optically active form. It should be understood that the invention encompasses all stereochemical isomeric forms, including diastereomeric, enantiomeric and epimeric forms, as well as d-Isomers and l-Isomers and mixtures thereof. Individual stereoisomers of compounds can be prepared synthetically from commercially available starting materials which contain chiral centres or by preparation of mixtures of enantiomeric products followed by separation such as conversion to a mixture of diastereomers followed by separation or recrystallization, chromatographic techniques, direct separation of enantiomers on chiral chromatographic columns or any other appropriate method known in the art. Starting compounds of particular stereochemistry are either commercially available or can be made and resolved by techniques known in the art. Additionally, the compounds of the present invention may exist as geometric Isomers. The present invention includes all cis, trans, syn, anti, entgegen (E) and zusammen (Z) Isomers as well as the appropriate mixtures thereof.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers. The disclosure includes enantiomers of the compounds described herein. Each compound herein disclosed includes all the enantiomers that conform to the general structure of the compound. The compounds may be in a racemic or enantiomerically pure form or any other form in terms of stereochemistry. In some embodiments the compounds are the (R, S)-enantiomer.

The term “diastereomers” refers to the set of stereoisomers which cannot be made superimposable by rotation around single bonds. For example, cis- and trans-double bonds, endo- and exo-substitution on bicyclic ring systems and compounds containing multiple stereogenic centres with different relative configurations are considered to be diastereomers. The term “diastereomer” refers to any member of this set of compounds. In some examples presented, the synthetic route may produce a single diastereomer or a mixture of diastereomers. The disclosure includes diastereomers of the compounds described herein.

The term “tautomer” refers to compounds in which hydrogen atoms are transposed to other parts of the molecules and the chemical bonds between the atoms of the molecules are consequently rearranged. Compounds of the present invention, free form and salts thereof, may exist in multiple tautomeric forms. It is understood that all tautomeric forms, insofar as they may exist, are included within the invention.

The compounds of the present invention may be used as single drug or as a pharmaceutical composition in which the compound is mixed with various pharmacologically acceptable excipients.

The compounds of the invention are typically administered in the form of a pharmaceutical composition. Such compositions can be prepared using procedures well known in the pharmaceutical art and comprise at least one compound of the invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents and solvents.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the patent application to the appropriate or desired site of action. Suitable routes of administration include, but are not limited to oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or haul gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present patent application may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present patent application.

### Experimental

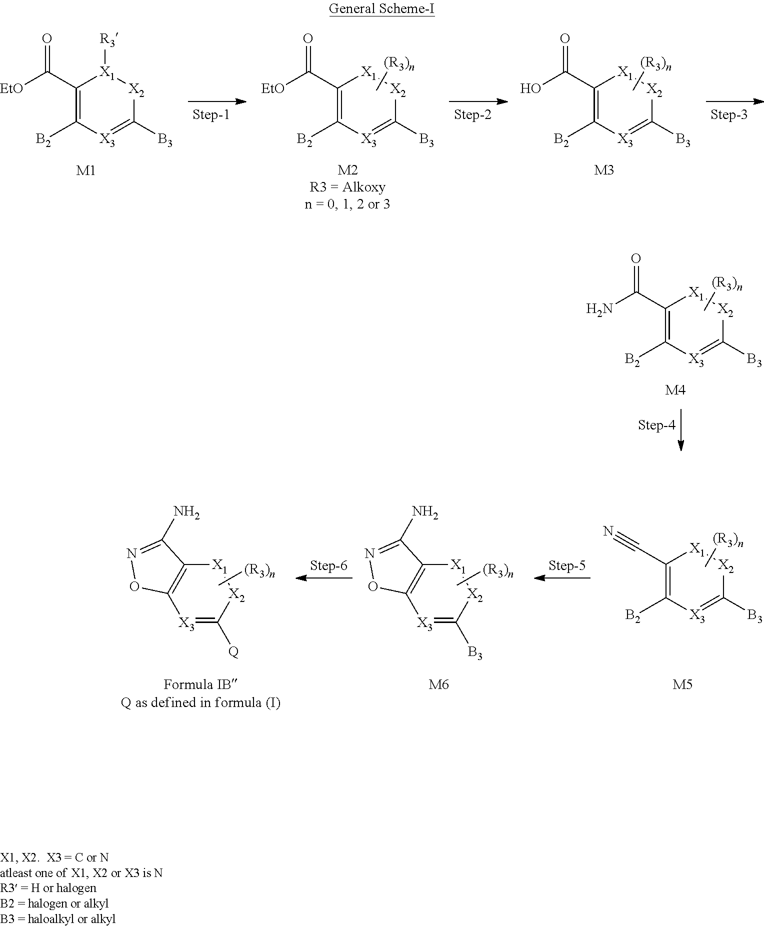

The general approach for the synthesis of compound of general formula (IB″) is depicted in the scheme below.

Compound of formula (M2) can be obtained from compound of formula (M1) by reacting with alkoxides in an appropriate solvent at suitable temperature. Compound of formula (M2), on hydrolysis in presence of a base in an appropriate solvent at suitable temperature can give compound of formula (M3) which upon further reaction with ammonia to yield compound of formula (M4). The compound of formula (M4) can undergo dehydration to give compound of formula (M5) which upon cyclization reaction with suitable amides in an appropriate solvent to give compound of formula (M6). The formula (M6) compound can undergo coupling reaction with suitable reagents to give compound of formula IB″.

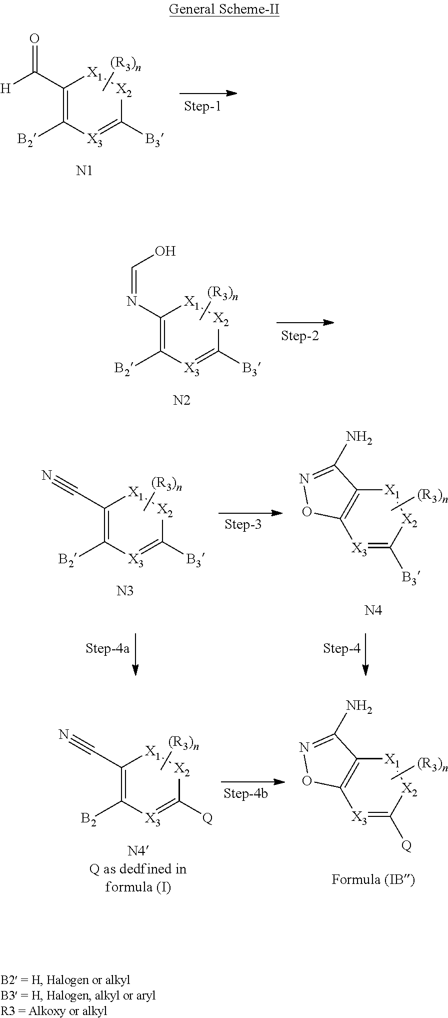

The general approach for the synthesis of compound of general formula (IB″) is depicted in the scheme below.

Compound of formula (N2) can be obtained from the corresponding aldehydes of formula (N1) by reacting with hydroxyl amine hydrochloride in an appropriate solvent. The compound of formula (N2) can undergo dehydration in the presence of suitable reagent and solvent to give compound of formula (N3) which can be cyclized in the presence of suitable reagent and solvent to give the compound of formula (N4). Upon coupling reaction with suitable reagents, compound of formula (N4) can yield compound of formula IB″. Alternatively, compound of formula (N3) can undergo coupling reaction in the presence of suitable reagent to give the compound of formula (N4′) which can be cyclized with suitable reagent in an appropriate solvent to give the compound of formula (IB″).

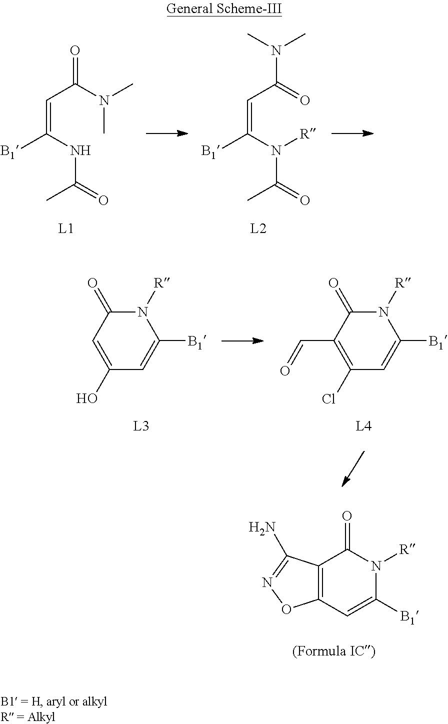

The general approach for the synthesis of compound of general formula (IC″) is depicted in the scheme below.

Compound of formula (L2) can be obtained from the compound of formula (L1) involving the alkylation reaction in an appropriate solvent at suitable temperature. The compound of formula (L2) can undergo cyclized in the presence of suitable reagent with appropriate solvent system to give the compound of formula (L3). The compound of formula (L3) oxidized with suitable reagents in an appropriate solvent to produce the compound of formula (L4) which is further cyclized in suitable solvent and reagent to produce the compound of formula (IC″).

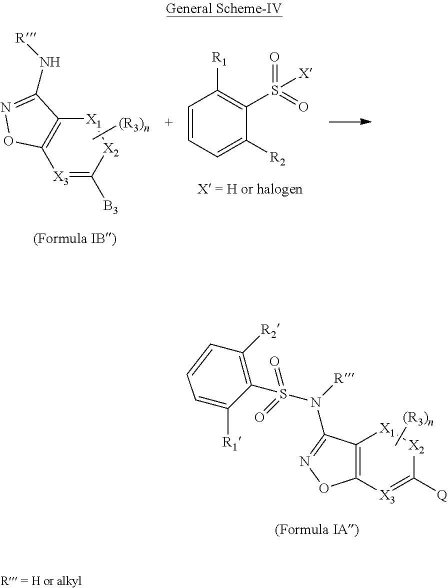

The general approach for the synthesis of compound of general formula (IA″) is depicted in the scheme below.

Compound of formula (IB″) can react with corresponding sulfonyl halides in an appropriate solvent can give compound of formula (IA″).

General Scheme-V The general approach for the synthesis of compounds of general formula (IA′″) is depicted in the scheme below.

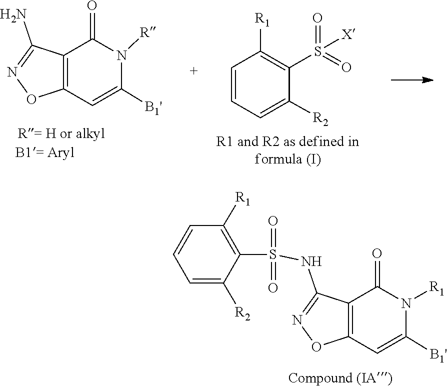

Compound of formula (IC″) can react with corresponding sulfonyl halides in an appropriate solvent can give compound of formula (IA′″).

The abbreviations used in the experimental refer to the definitions below respectively:

DMSO—Dimethylsulfoxide; DIPEA—N,N-Diisopropylethylamine; THF—Tetrahydrofuran; DCM-Dichloromethane; 1,2-DCE—1,2-Dichloroethane; K₂CO₃—Potassium carbonate; LiHMDS—Lithium bis(trimethylsilyl)amide; TEA—Triethyl amine; AgF₂—silver difluoride; NaH—sodium hydride; K₃PO₄—Potassium carbonate; DMAP—Dimethylaminopyridine; POCl₃—Phosphorus(V) oxychloride; Pd(DPPF)Cl₂·DCM—[1,1′-Bis(diphenylphosphino)ferrocene]dichloropalladium(II) dichloromethane Complex; Pd(Amphos)Cl₂—Bis(di-tert-butyl(4-dimethylaminophenyl)phosphine)dichloropalladium(II); NH₄Cl—Ammonium chloride; Na₂SO₄—Sodium sulphate; br—Broad; ° C.—Degree Celsius; DMSO-d6—Deuterated dimethylsulfoxide; DMF—N, N-Dimethylformamide; g—Gram; h—Hours; 1H—Proton; LC-MS—Liquid Chromatography—Mass Spectroscopy; MHz—Mega Hertz (frequency); MS—Mass Spectroscopy; M—Molar; mmol—Milli Mole; mL—Milli Litre; min—Minutes; mol—Moles; M^{+/-}—Molecular ion; m/z—mass to charge ratio; NMR—Nuclear Magnetic Resonance; ppm—Parts per million; rt/RT—Room temperature; RM—Reaction mixture; s—Singlet; d—Doublet, t—Triplet; q—Quartet; m—Multiplet; dd—doublet of doublets; TLC—Thin Layer Chromatography; %—Percentage and δ—Delta.

### Synthesis of Intermediates

### Intermediate-1: 6-Chloro-4-methoxyisoxazolo[5,4-b]pyridin-3-amine

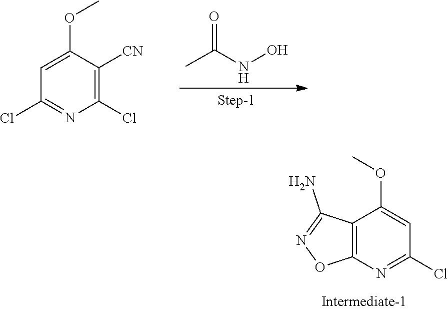

To a solution of 2,6-dichloro-4-methoxynicotinonitrile (1 g, 4.92 mmol) (prepared as explained in WO2018106459) in acetonitrile (16 mL) and water (4 mL), was added N-hydroxy acetamide (0.37 g, 4.92 mmol) followed by K₂CO₃ (0.681 g, 4.92 mmol). The reaction mixture was stirred at 30° C. for 3 h, then the reaction mixture was extracted into DCM. The organic portion was separated, dried over Na₂SO₄ and concentrated to get crude mass. The crude was further purified by combiflash chromatography using 75% ethyl acetate in hexane as eluent (0.169 g, 17.1%). LC-MS: 200.0 [M+H]⁺

### Intermediate-2: 6-Phenylisoxazolo[4,5-b]pyridin-3-amine

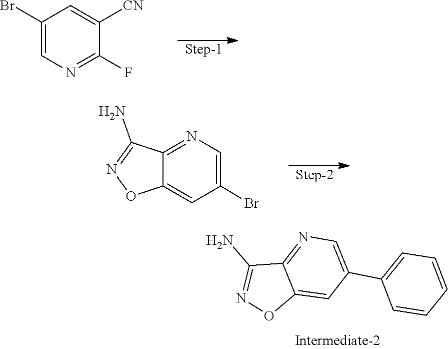

### Step-1: Synthesis of 6-bromoisoxazolo[4,5-b]pyridin-3-amine

To a solution of 5-bromo-2-fluoronicotinonitrile (2 g, 9.95 mmol), acetohydroxamic acid (0.822 g, 10.9 mmol) and K₂CO₃ (2.7 g, 19.9 mmol) in water (10 mL), were stirred at RT for 12 h. The reaction mixture was extracted into ethyl acetate, then the organic portion was dried over Na₂SO₄ and concentrated to get the crude solid. The crude was purified by Combiflash chromatography by eluting with 0-20% ethyl acetate in hexane as eluent which afforded the title compound (1.3 g, 61.05%) LC-MS: 215.9 [M+2H]⁺

### Step-2: Synthesis of 6-phenylisoxazolo[4,5-b]pyridin-3-amine

To a solution of 6-bromoisoxazolo[4,5-b]pyridin-3-amine (0.4 g, 1.86 mmol) in 1,4-dioxane (4 mL) and water (1 mL), were added phenyl boronic acid (0.34 g, 2.8 mmol) and K₂CO₃ (0.77 g, 5.6 mmol). The reaction mixture was purging with nitrogen gas for 15 min, then Pd(Amphos)Cl₂ was added and the reaction mixture was heated to 100° C. for 12 h. The reaction mixture was filtered through Celite® and organic portion was washed with water, dried over Na₂SO₄ and concentrated to get crude compound. The crude was further purified in Combiflash chromatography using 40% ethyl acetate in hexane (0.33 g, 83.59%) LC-MS: 212.0 [M+H]⁺

### Intermediate-3: 6-Phenylisoxazolo[4,5-c]pyridin-3-amine

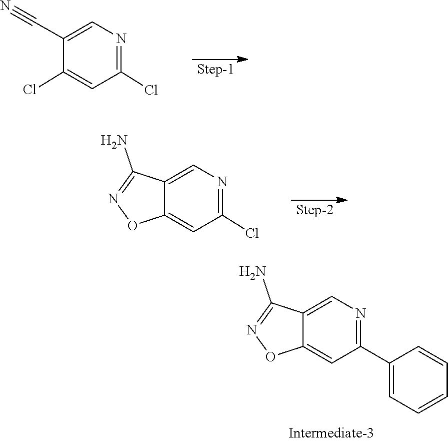

Intermediate-3 was prepared according to the procedure described in the synthesis of Intermediate-2 with appropriate variations in coupling methods, reactants, quantities of reagents and solvents. LC-MS: 212.2 [M+H]⁺

### Intermediate-4: 4-Chloro-2-methoxy-6-phenylpyridine

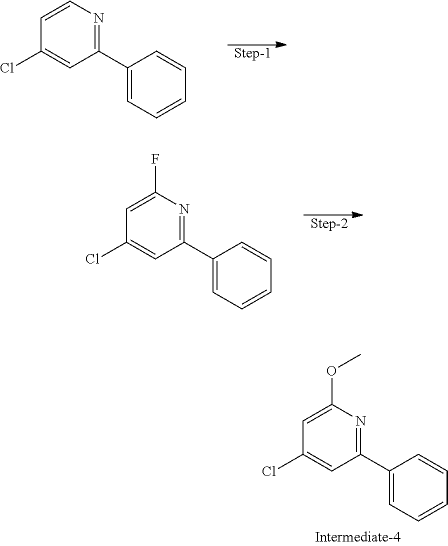

### Step-1: Synthesis of 4-chloro-2-fluoro-6-phenylpyridine

To a solution of 4-chloro-2-phenylpyridine (3.0 g, 15.8 mmol) in acetonitrile (30 mL) was added AgF₂ (6.92 g, 47.4 mmol) were taken in a sealed tube. The reaction mixture was stirred at 70° C. for 12 h. Then the reaction mixture was passed through Celite® bed. The reaction mixture was washed with ethyl acetate and concentrated to get the residue. The residue was purified by Combiflash chromatography using 5% ethyl acetate in hexane as solvent. This afforded title compound (1.1 g, 33.49%). 1H-NMR (400 MHz, DMSO-D6) δ: 7.97-7.94 (m, 2H), 7.61 (s, 1H), 7.48-7.43 (m, 3H), 7.24-6.87 (m, 1H).

### Step-2: Synthesis of 4-chloro-2-methoxy-6-phenylpyridine

A stirred solution of 4-chloro-2-fluoro-6-phenylpyridine (1 g, 4.81 mmol) in methanol (10 mL) was added to sodium methoxide (0.78 g, 51.02 mmol) at 0° C. The reaction mixture was gradually warmed to RT and stirred for 16 h. Then the reaction mixture was concentrated to get crude mass. The crude was purified by Combiflash chromatography using hexane as eluent to get the pure title compound (0.9 g, 85.07%). LC-MS: 220.0 [M+H]⁺

### Intermediate-5: 4-Chloro-1-methyl-2-oxo-6-phenyl-1,2-dihydropyridine-3-carbaldehyde

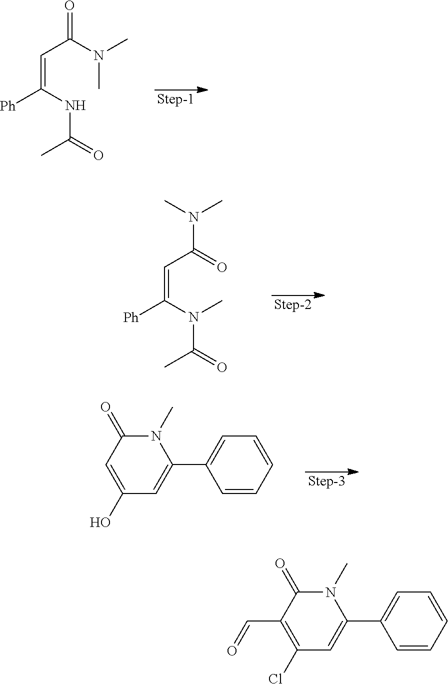

### Step-1: Synthesis of (Z)—N,N-dimethyl-3-(N-methylacetamido)-3-phenylacrylamide

To a solution of (Z)-3-acetamido-N,N-dimethyl-3-phenylacrylamide (4.5 g, 19.3 mmol) (Prepared as explained in ACS Catalysis, 9(9), 8128-8135; 2019) in DMF was added NaH (1.39 g, 58.1 mmol) at 0° C. and stirred for 15 min. Then methyl iodide (5.5 g, 38.7 mmol) was added to the reaction mixture. The reaction mixture was gradually warmed at RT and allowed to stirred for 12 h. The reaction mixture was diluted with ethyl acetate and organic portion was washed with water and brine solution, dried over Na₂SO₄ and concentrated to get crude compound. The crude was purified by Combiflash chromatography by eluting with 0-4% methanol in DCM. This afforded the pure title compound (2 g, 41.9%) LC-MS: 247.1 [M+H]⁺

### Step-2: Synthesis of 4-hydroxy-1-methyl-6-phenylpyridin-2(1H)-one

To a solution of (Z)—N, N-dimethyl-3-(N-methylacetamido)-3-phenylacrylamide (2.5 g, 1.15 mmol) in anhydrous THE (25 mL) was added LiHMDS (30.4 mL, 1M Solution) dropwise at 0° C. The reaction mixture was then warmed at RT, heated to 55° C. for 3 h and quenching with saturated NH₄Cl solution. Then the mixture was extracted with ethyl acetate and concentrated. The crude was purified by Combi flash chromatography using 0-5% methanol in DCM. This afforded the title compound (0.7 g, 34.2%). LC-MS: 202.0 [M+H]⁺

### Step-3: Synthesis of 4-Chloro-1-methyl-2-oxo-6-phenyl-1,2-dihydropyridine-3-carbaldehyde

To a solution of 4-hydroxy-1-methyl-6-phenylpyridin-2(1H)-one (1 g, 4.9 mmol) in DMF (10 mL) was dropwise added POCl₃ (1.37 g, 8.9 mmol). The reaction mixture was heated at 60° C. for 3 h. The reaction mixture was then cooled to RT and quenched into ice cold water. Then the reaction mixture was extracted with ethyl acetate. The organic portion was washed with water and brine solution, dried over Na₂SO₄ and concentrated. The residue was purified by Combiflash chromatography using 0-5% methanol in DCM as eluent to give pure title compound (1 g, 81.2%). LC-MS: 248.1 [M+H]⁺

### Intermediate-6: 4-Methoxy-6-phenylisoxazolo[5,4-b]pyridin-3-amine

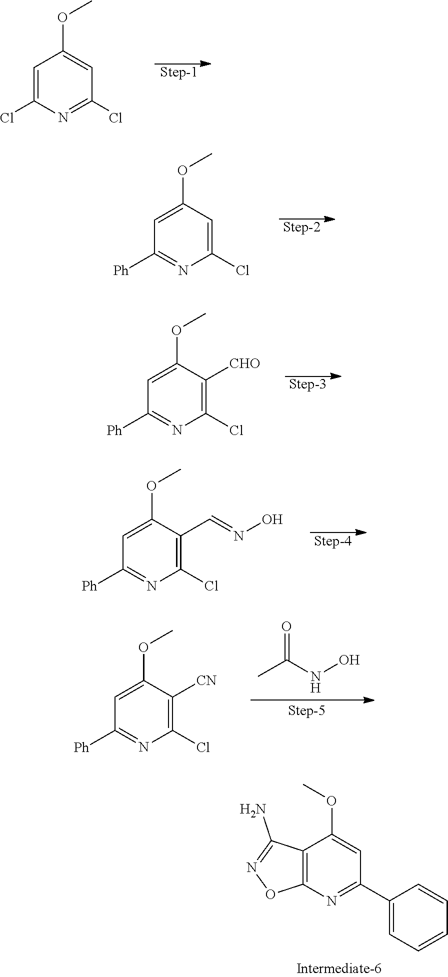

### Step-1: Synthesis of 2-chloro-4-methoxy-6-phenylpyridine

To a solution of 2,6-dichloro-4-methoxypyridine (5 g, 28.08 mmol) in 1,4-dioxane (75 mL) and water (19 mL) were added phenylboronic acid (3.42 g, 19.66 mmol) and K₃PO₄ (17.8 g, 84.2 mmol). The reaction mixture was purged with nitrogen, then Pd(DPPF)Cl₂·DCM (2.2 g, 2.8 mmol) was added to the reaction mixture. The resultant reaction mixture was heated at 80° C. for 6 h. Then the reaction mixture was cooled to RT and filtered through Celite® followed by washed with ethyl acetate. The filtrate was washed with water and brine solution. The organic portion was dried over Na₂SO₄ and concentrated to get crude compound. The crude was further purified using Combiflash column chromatography using 0-20% DCM in hexane as eluent to obtain pure title compound (3 g, 48.6%). LC-MS: 220.1[M+H]⁺

### Step-2: Synthesis of 2-chloro-4-methoxy-6-phenylnicotinaldehyde

To a solution of 2-chloro-4-methoxy-6-phenylpyridine (4.5 g, 20.4 mmol) in dry THF (80 mL) was cooled to −78° C. To this, n-butyllithium (30.72 mmol, 1.5 Eq) was added dropwise over a period of 10 min followed by DMF (2.9 g, 40.9 mmol) in THF (10 mL) was added over a period of 5 min. The resultant mixture was stirred at −78° C. for 2 h and quenched with saturated NH₄Cl solution and extracted with ethyl acetate, then dried over sodium sulphate and concentrated. The crude was purified using Combiflash chromatography by eluting with 10-15% ethyl acetate in hexane. This afforded title compound (3.0 g, 59.13%). LC-MS: 248.1[M+H]⁺

### Step-3: Synthesis of (E)-2-chloro-4-methoxy-6-phenylnicotinaldehyde oxime

To a solution of 2-chloro-4-methoxy-6-phenylnicotinaldehyde (3.0 g, 12.12 mmol) in THF (60 mL) were added hydroxylamine hydrochloride (0.926 g, 13.2 mmol) and DIPEA (2.3 g, 18.1 mmol) at 0° C. The reaction mixture was gradually warmed to RT and stirred for 12 h. The reaction mixture was diluted with ethyl acetate and washed with water, dried over Na₂SO₄ and concentrated to get the title compound (3 g, 94.29%). LC-MS: 263.1 [M+H]⁺

### Step-4: Synthesis of 2-chloro-4-methoxy-6-phenylnicotinonitrile

To a solution of (E)-2-chloro-4-methoxy-6-phenylnicotinaldehyde oxime (3.0 g, 11.4 mmol) and trimethylamine (4.6 g, 45.6 mmol) in 1,2-DCE (30 mL) was dropwise added trifluoroacetic anhydride (4.7 g, 22.8 mmol) at 0° C. After completion of the addition, the reaction mixture was stirred at RT for 2 h. Then the reaction mixture was added to ice cold water and extracted into DCM. The organic portion was dried over Na₂SO₄ and concentrated to get the title compound (2.7 g, 96.63%). LC-MS: 245.1[M+H]⁺

### Step-5: Synthesis of 4-methoxy-6-phenylisoxazolo[5,4-b]pyridin-3-amine

To a degassed solution of 2-chloro-4-methoxy-6-phenylnicotinonitrile (2.7 g, 11.03 mmol), N-hydroxyacetamide (2.76 g, 36.78 mmol) in acetonitrile (31 mL) and water (5 mL), was added K₂CO₃ (10.1 g, 73.5 mmol). The reaction mixture was stirred at 70° C. for 12 h. Then the reaction mixture was diluted with ethyl acetate. The organic portion was washed with water and brine solution, dried over Na₂SO₄ and concentrated to get the crude compound. The crude was further purified by Combiflash chromatography using 30% ethyl acetate in hexane as eluent. This afforded title compound (1.2 g, 45.08%). LC-MS: 242.1[M+H]⁺

The below intermediates (Intermediate-7 to Intermediate-9) were prepared according to the procedure described in the synthesis of Intermediate-6 with appropriate variations in coupling methods, reactants, quantities of reagents and solvents.

| Intermediate | | Starting | Spectral |
|---|---|---|---|
| No. | Structure | Material | data [M + H]⁺ |
| 7 | 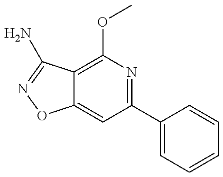 [SMILES: COc1nc(-c2ccccc2)cc2onc(N)c12] | 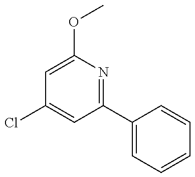 | LC-MS: 242.2 |
| | | Intermediate-4 | |
| 8 | 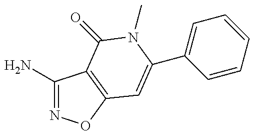 [SMILES: Cn1c(-c2ccccc2)cc2onc(N)c2c1=O] | 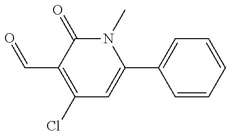 | LC-MS: 242.05 |
| | | Intermediate-5 | |
| 9 | 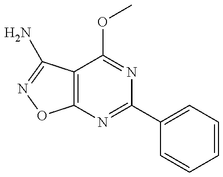 [SMILES: COc1nc(-c2ccccc2)nc2onc(N)c12] | 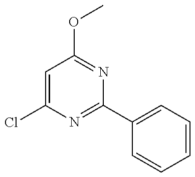 | LC-MS: 243.1 |

### Intermediate-10: 4-Methoxy-6-(pyrrolidin-1-yl)isoxazolo[5,4-b]pyridin-3-amine

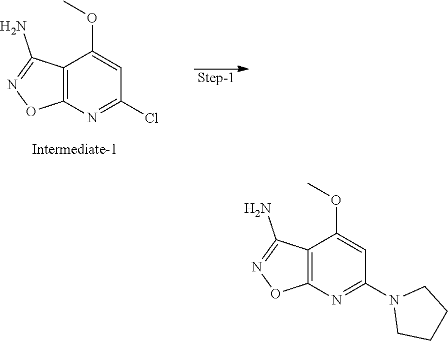 [SMILES: COc1cc(Cl)nc2onc(N)c12 ; COc1cc(N2CCCC2)nc2onc(N)c12]

### Step-1: Synthesis of 4-methoxy-6-(pyrrolidin-1-yl)isoxazolo[5,4-b]pyridin-3-amine

To a solution of 6-chloro-4-methoxyisoxazolo[5,4-b]pyridin-3-amine (Intermediate 1, 0.05 g, 0.251 mmol) in dry THF (1 mL), was added pyrrolidine (0.081 g, 0.75 mmol) and heated at 50° C. for 12 h. Then the solvent was evaporated off to get the crude. The crude was purified by preparative TLC using 5% methanol in DCM as eluent which afforded the title compound (0.04 g, 68.03%). LC-MS: 235.1 [M+H]⁺

The below intermediates (Intermediate-11 to Intermediate-17) were prepared according to the procedure described in the synthesis of Intermediate-10 with appropriate variations in coupling methods, reactants, quantities of reagents and solvents.

| | | | | Spectral data |
|---|---|---|---|---|
| Intermediate | | | | LC-MS: |
| No. | Structure | Starting Materials | | [M + H]⁺ |
| 11 | 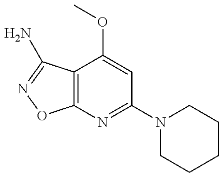 [SMILES: COc1cc(N2CCCCC2)nc2onc(N)c12] | 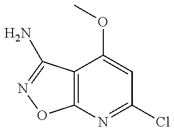 | Piperidine | 249.1 |
| 12 | 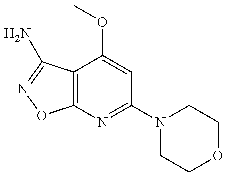 [SMILES: COc1cc(N2CCOCC2)nc2onc(N)c12] | 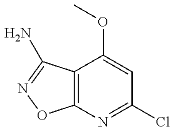 | Morpholine | 251.1 |
| 13 | 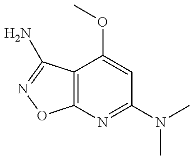 | 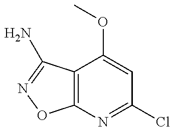 | Dimethylamine | 208.2 |
| 14 | 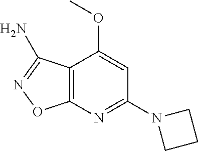 [SMILES: COc1cc(N2CCC2)nc2onc(N)c12] | 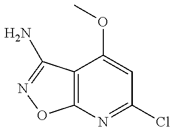 | azetidine hydrochloride | 221.1 |
| 15 | 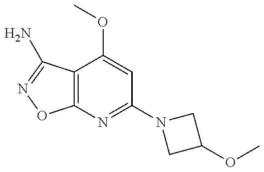 [SMILES: COc1cc(N2CC(OC)C2)nc2onc(N)c12] | 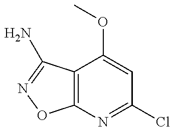 | 3-methoxyazetidine hydrochloride | 251.05 |
| 16 | 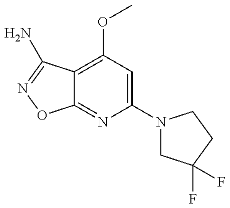 [SMILES: COc1cc(N2CCC(F)(F)C2)nc2onc(N)c12] | 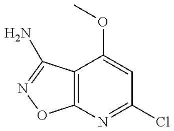 | 3,3- difluoropyrrolidine Hydrochloride | 271.0 |
| 17 | 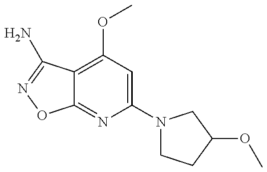 [SMILES: COc1cc(N2CCC(OC)C2)nc2onc(N)c12] | 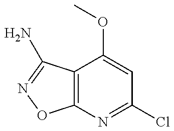 | 3- methoxypyrrolidine hydrochloride | 265.05 |
| 18 | 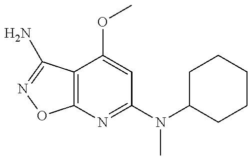 [SMILES: COc1cc(N(C)C2CCCCC2)nc2onc(N)c12] | 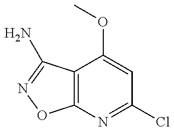 | N- methylcyclo- hexanamine | 277.1 |
| 19 | 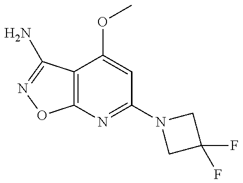 [SMILES: COc1cc(N2CC(F)(F)C2)nc2onc(N)c12] | 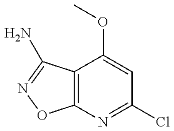 | 3,3- difluoroazetidine | 257.1 |
| 20 | 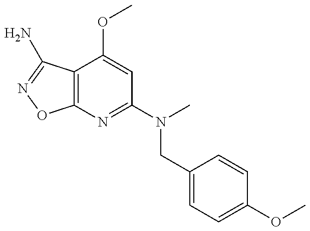 [SMILES: COc1ccc(CN(C)c2cc(OC)c3c(N)noc3n2)cc1] | 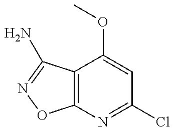 | 1-(4- methoxyphenyl)-N- methylmethanamine | 315.20 |
| 21 | 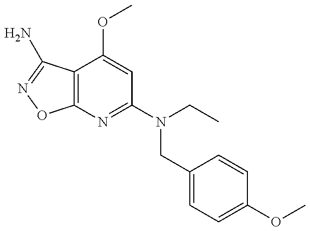 [SMILES: CCN(Cc1ccc(OC)cc1)c1cc(OC)c2c(N)noc2n1] | 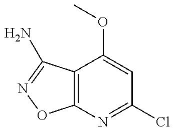 | N-(4- methoxybenzyl) ethanamine | 329.30 |

### Intermediate-22: 4-Methoxy-6-(2-methoxyphenyl)isoxazolo[5,4-b]pyridin-3-amine

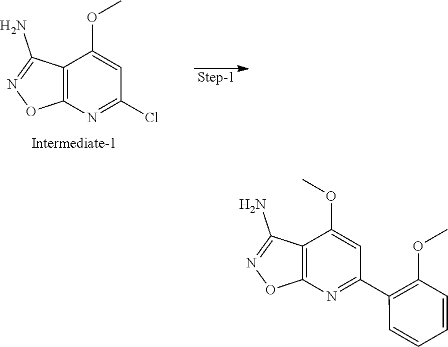 [SMILES: COc1cc(Cl)nc2onc(N)c12 ; COc1ccccc1-c1cc(OC)c2c(N)noc2n1]

To a solution of 6-chloro-4-methoxyisoxazolo[5,4-b]pyridin-3-amine (0.1 g, 501 mmol) and 2-(2-methoxyphenyl)-4,4,5,5-tetramethyl-1,3,2-dioxaborolane (141 mg, 0.601 mmol) in 1,4-dioxane (4 mL) and water (1 mL) was added K₂CO₃ (208 mg, 1.5 mmol) and purged with nitrogen then added Pd(Amphos)Cl₂ (0.035 g, 0.050 mmol). The reaction mixture was stirred at 100° C. for 12 h. Then the reaction mixture was diluted with 10% methanol in DCM, dried over Na₂SO₄ and concentrated to get crude. The crude was passed in a flash column using 5% Methanol ion DCM to get title compound (100 mg). LC-MS: 272.2 [M+H]⁺

### Intermediate 23: 4-methoxy-6-(thiophen-3-yl)isoxazolo[5,4-b]pyridin-3-amine

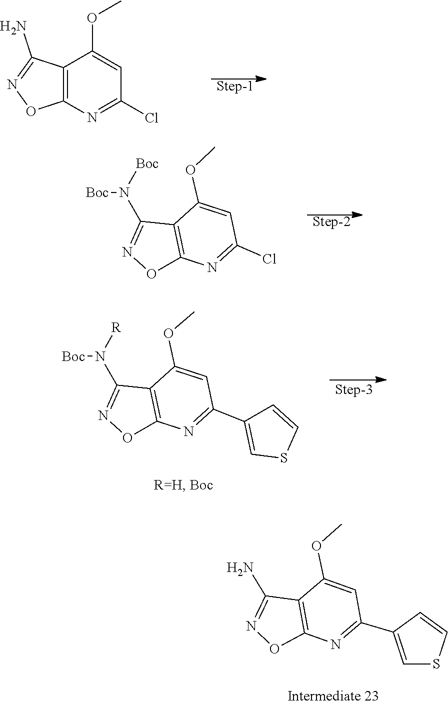

### Step-1: Synthesis of tert-butyl(tert-butoxycarbonyl)(6-chloro-4-methoxyisoxazolo[5,4-b]pyridin-3-yl)carbamate

To a solution of intermediate-1 (0.500 g, 2.50 mmol) in DCM (5.0 mL) was added TEA (0.760 g, 7.51 mmol) and DMAP (0.031 g, 0.25 mmol) at 0° C., followed by di-tert-butyl decarbonate (2.732 g, 12.52 mmol). The resulting reaction mixture was stirred at RT for 16 h. The reaction mixture was diluted with water and extracted using DCM. The combined organic layers were washed with water and brine, dried over sodium sulphate, filtered, concentrated, and purified by flash chromatography using 20-40% ethyl acetate in hexane as eluent to afford the title compound (0.500 g, 49.94%). LC-MS: 400.2 [M+H]⁺

### Step-2: Synthesis of tert-butyl (tert-butoxycarbonyl)(4-methoxy-6-(thiophen-3-yl)isoxazolo[5,4-b]pyridin-3-yl)carbamate

To a degassed solution of tert-butyl (tert-butoxycarbonyl)(6-chloro-4-methoxyisoxazolo[5,4-b]pyridin-3-yl)carbamate (0.200 g, 0.5 mmol), K₃PO₄ (0.318 g, 1.5 mmol), and thiophen-3-ylboronic acid (0.128 g, 1 mmol) in 1 4-dioxane (2.5 mL) and water (0.5 mL) was added Pd(amphos)Cl₂ (0.004 g, 0.005 mmol) and stirred at 90° C. for 2 h. After completion of the reaction, the reaction mixture was diluted with 10% MeOH-DCM and filtered over Celite. The filtrate was concentrated under vacuum and purified by flash chromatography using 30-70% ethyl acetate in hexane as eluent to afford the title compound (0.12 g, 53.63%). LC-MS: 345.9 [M−H]⁻.

### Step-3: Synthesis of 4-methoxy-6-(thiophen-3-yl)isoxazolo[5,4-b]pyridin-3-amine

To a solution of tert-butyl (tert-butoxycarbonyl)(4-methoxy-6-(thiophen-3-yl)isoxazolo[5,4-b]pyridin-3-yl)carbamate (0.120 g, 0.268 mmol) in DCM (3.0 mL) was added 4M HCl in dioxane (0.67 mL, 2.68 mmol) and stir the reaction mixture at RT for 4 h. The reaction mixture was concentrated, basified using aqueous NaHCO₃ and extracted with DCM/MeOH. The combined organic layers were dried over sodium sulfate, filtered, concentrated, to afford the crude compound. The crude was washed with diethyl ether and filtered to afford tittle compound. LC-MS: 248.1 [M+H]⁺

### Intermediate-24: Synthesis of 6-(cyclopent-1-en-1-yl)-4-methoxyisoxazolo[5,4-b]pyridin-3-amine

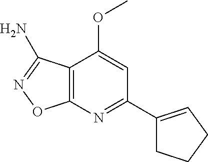 [SMILES: COc1cc(C2=CCCC2)nc2onc(N)c12]

The below Intermediate-24 was prepared according to the procedure described in the synthesis of Intermediate-23 with appropriate variations in coupling methods, reactants, quantities of reagents and solvents. LC-MS: 232.1 [M+H]⁺

### Intermediate-25: Synthesis of 6-((1H-pyrazol-1-yl)methyl)-4-methoxyisoxazolo[5,4-b]pyridin-3-amine

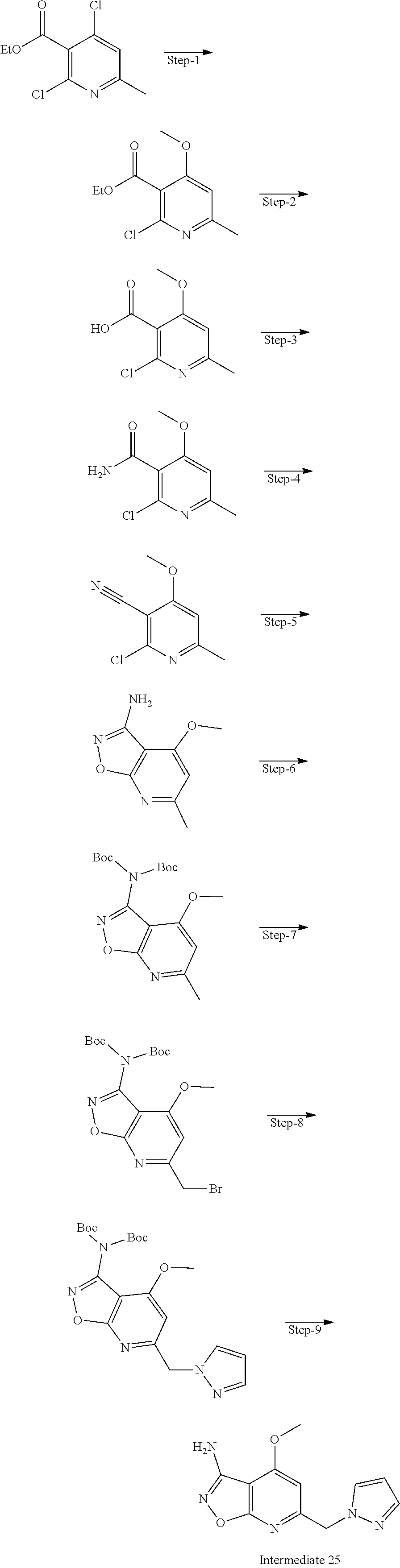

### Step-1: Synthesis of ethyl 2-chloro-4-methoxy-6-methylnicotinate

To a solution of ethyl2,4-dichloro-6-methylnicotinate (15 g, 64.08 mmol) in methanol (150.0 mL) was added sodium methanolate (3.80 g, 70.48 mmol) and stirred at 50° C. for 12 h. The reaction mixture was diluted with water and extracted with ethyl acetate. The combined organic layer was dried over Na₂SO₄, followed by filtered, concentrated and purified by flash chromatography using 0-70% ethyl acetate in hexane as eluent to afford the title compound (11.1 g, 74.74%). LC-MS: 230.1 [M+H]⁺

### Step-2: Synthesis of 2-Chloro-4-methoxy-6-methylnicotinic acid

To a solution of ethyl 2-chloro-4-methoxy-6-methylnicotinate (11.0 g, 47.9 mmol) in THF (55 mL), MeOH (33 mL) and water (22 mL) was added LiOH·H₂O (3.01 g, 71.84 mmol) and refluxed at 60° C. for 16 h. The reaction mixture was cooled to RT, acidified with Amberlyst-15 and filtered through celite. The filtrate was contracted to obtain the title compound as white solid (9.5 g, 98.38%). LC-MS: 202.20 [M+H]⁺.

### Step-3: Synthesis of 2-Chloro-4-methoxy-6-methylnicotinamide

To a solution of 2-chloro-4-methoxy-6-methylnicotinic acid (9.5 g, 47.12 mmol) in THF (90 mL) was added thionyl dichloride (19.95 g, 167.75 mmol) at 0° C. Then the reaction mixture was refluxed at 80° C. for 2 h. The reaction mixture was cooled to RT and concentrated in rotavapor. The crude reaction mixture was dissolved in DCM and cooled at 0° C. Aqueous ammonia (587.18 mmol) was added dropwise to this cooled reaction mixture and stirred for 15 min. The reaction mixture was filtered and the solid obtained was washed with water twice and dried under vacuum to obtain the title compound (5.0 g, 52.89%). LC-MS: 201.1 [M+H]⁺.

### Step-4: Synthesis of 2-Chloro-4-methoxy-6-methylnicotinonitrile

To a solution of 2-chloro-4-methoxy-6-methylnicotinamide (4.8 g, 23.92 mmol) in DCE (20 mL) was added thionyl dichloride (19.18 g, 161.26 mmol) at 0° C. and refluxed at 85° C. for 12 h. After the reaction completion, the reaction mixture was concentrated. The residue was dissolved in DCM, washed with Sodium bicarbonate and brine. The combined organic layer was dried over Na₂SO₄, filtered and concentrated. The crude was recrystallized in n-pentane to provide the pure compound (3.6 g, 82.40%) LC-MS: 180.1 [M+H]⁺.

### Step-5: Synthesis of 4-Methoxy-6-methylisoxazolo[5,4-b]pyridin-3-amine

To a solution of 2-chloro-4-methoxy-6-methylnicotinonitrile (3.6 g, 19.71 mmol) in acetonitrile (5 mL) and water (20 mL) were added N-hydroxy acetamide (1.48 g, 19.71 mmol) and potassium carbonate (5.44 g, 39.42 mmol). The reaction mixture was refluxed at 70° C. for 12 h. Then the reaction mixture was concentrated, followed by diluted with water and extracted with ethyl acetate. The combined organic layers were dried over Na₂SO₄, filtered, concentrated and purified by flash chromatography using 0-70% ethyl acetate in hexane as eluent to afford the title compound (3.3 g, 93.42%). LC-MS: 180.1 [M+H]⁺

### Step-6: Synthesis of tert-butyl (tert-butoxycarbonyl)(4-methoxy-6-methylisoxazolo[5,4-b]pyridin-3-yl)carbamate

To a solution of 4-methoxy-6-methylisoxazolo[5,4-b]pyridin-3-amine (3.3 g, 18.41 mmol) in DCM (66.0 mL) was added TEA (2.26 g, 18.14 mmol) at 0° C. followed by addition of di-tert-butyl decarbonate (10.04 g, 46.04 mmol) and stirred at RT for 16 h. The reaction mixture was diluted with water and extracted using DCM. The combined organic layer was dried over solid sodium sulphate, filtered, concentrated, and purified by flash chromatography using 20-40% ethyl acetate in hexane as eluent to afford the title compound (4.5 g, 64.40%). LC-MS: 380.1 [M+H]⁺

### Step-7: Synthesis of tert-butyl (6-(bromomethyl)-4-methoxyisoxazolo[5,4-b]pyridin-3-yl)(tert-butoxycarbonyl)carbamate

To a solution of tert-butyl (tert-butoxycarbonyl)(4-methoxy-6-methylisoxazolo[5,4-b]pyridin-3-yl)carbamate (4.5 g, 11.86 mmol) in CCl₄ (100 mL) was added 2-(2-cyanopropan-2-yldiazenyl)-2-methylpropanenitrile (0.108 g, 0.650 mmol) followed by N-bromo succinimide (2.58 g, 14.49 mmol) at RT. The resulting reaction mixture was heated at 80° C. for 2 h. After the reaction completion, the reaction mixture was extracted with ethyl acetate. The combined organic layer was dried over Na₂SO₄, filtered, concentrated and purified by flash chromatography using 0-70% ethyl acetate in hexane as eluent to afford the title compound (2.2 g, 40.47%). LC-MS: 458.0 [M+H]⁺

### Step-8: Synthesis of tert-butyl (6-((1H-pyrazol-1-yl)methyl)-4-methoxyisoxazolo[5,4-b]pyridin-3-yl)(tert-butoxycarbonyl)carbamate

To a solution of tert-butyl (6-(bromomethyl)-4-methoxyisoxazolo[5,4-b]pyridin-3-yl)(tert-butoxycarbonyl)carbamate (1.0 g, 2.18 mmol) and pyrazole (0.446 g, 6.54 mmol) in THF (20 mL) was added Cs₂CO₃ (0.711 g, 2.18 mmol) at room temperature. Then the reaction mixture was stirred at RT for 2 h. The reaction mixture was diluted with water and extracted with EtOAc. The combined organic layers were washed with brine, dried over sodium sulphate, then filtered, concentrated and purified by flash chromatography using 0-70% ethyl acetate in hexane as eluent to afford the title compound (0.9 g, 92.59%). LC-MS: 446.30 [M+H]⁺

### Step-9: Synthesis of 6-((1H-pyrazol-1-yl)methyl)-4-methoxyisoxazolo[5,4-b]pyridin-3-amine

To a solution of tert-butyl (6-((1H-pyrazol-1-yl)methyl)-4-methoxyisoxazolo[5,4-b]pyridin-3-yl)(tert-butoxycarbonyl)carbamate (0.90 g, 2.02 mmol) in DCM (10.0 mL) was added HCl (1.5 mL, 6.0 mmol, 4M in dioxane) and stir the reaction mixture at RT for 2 h. The reaction mixture was concentrated, the obtained residue was basified using aqueous NaHCO₃ and extracted with DCM/MeOH. The combined organic layer was dried over sodium sulfate, filtered, concentrated, and purified by flash chromatography using 0-70% ethyl acetate in hexane as eluent to afford the title compound (0.250 g, 50.47%). LC-MS: 246.10 [M+H]⁺

### EXAMPLES

### Example-1: 2,6-dimethoxy-N-(4-methoxy-6-phenylisoxazolo[5,4-b]pyridin-3-yl)benzenesulfonamide (Compound-1)

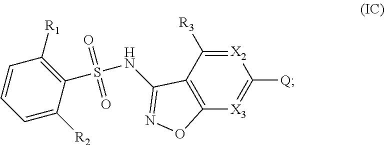

To a suspension of 4-methoxy-6-phenylisoxazolo[5,4-b]pyridin-3-amine (0.3 g, 1.24 mmol), 2,6-dimethoxybenzenesulfonyl chloride (0.353 g, 1.49 mmol) in THF (10 mL), was added LiHMDS (3.7 mL 1M Solution in THF) at 0° C. and stirred at the same temperature for 10 min. The reaction mixture was then quenched with NH₄Cl and diluted with ethyl acetate. The organic portion was washed with water and brine, dried over sodium sulphate and concentrated to get the crude. The crude was purified by using preparative TLC using 2% methanol in DCM as eluent. Further, the obtained material was washed with diethyl ether to give pure title compound (0.16 g, 29.1%). LC-MS: 442.2 [M+H]⁺; 1H-NMR (400 MHz, DMSO-D6) δ 10.37 (s, 1H), 8.19-8.18 (m, 2H), 7.54-7.49 (m, 5H), 6.80-6.78 (d, 2H), 4.07 (s, 3H), 3.78 (s, 6H).

The below compounds (2 to 24) were prepared according to the procedure described in the synthesis of Example-1 with appropriate variations in coupling methods, reactants, quantities of reagents and solvents.

| Com- | | | |
|---|---|---|---|
| pound | Structure | Intermediate | Spectral data |
| 2 | 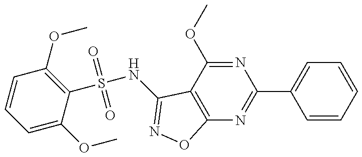 [SMILES: COc1cccc(OC)c1S(=O)(=O)Nc1noc2nc(-c3ccccc3)nc(OC)c12] | 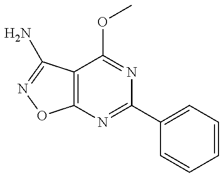 | LC-MS: 443.0 [M + H]⁺; 1H- NMR (400 MHz, DMSO-D6) δ 8.47-8.45 (d, 2H), 7.61-7.50 (m, 4H), 6.81- 6.79 (d, 2H), 4.17 (s, 3H), 3.78 (s, 6H). |
| | 2,6-dimethoxy-N-(4- | Intermediate-9 | |
| | methoxy-6- | | |
| | phenylisoxazolo[5,4- | | |
| | d]pyrimidin-3- | | |
| | yl)benzenesulfonamide | | |
| 3 | 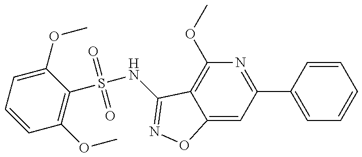 [SMILES: COc1cccc(OC)c1S(=O)(=O)Nc1noc2cc(-c3ccccc3)nc(OC)c12] | 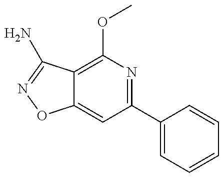 | LC-MS: 442.2 [M + H]⁺; 1H- NMR (400 MHz, DMSO-D6) δ 8.21-8.19 (d, 2H), 7.92 (s, 1H), 7.53-7.50 (m, 4H), 6.80- 6.78 (d, 2H), 4.09 (s, 3H), 3.78(s, 6H). |
| | 2,6-dimethoxy-N-(4- | Intermediate-7 | |
| | methoxy-6- | | |
| | phenylisoxazolo[4,5- | | |
| | c]pyridin-3- | | |
| | yl)benzenesulfonamide | | |
| 4 | 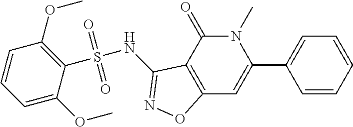 [SMILES: COc1cccc(OC)c1S(=O)(=O)Nc1noc2cc(-c3ccccc3)n(C)c(=O)c12] | 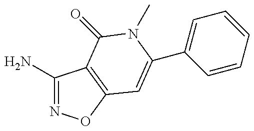 | LC-MS: 442.0 [M + H]⁺; 1H- NMR (400 MHz, DMSO-D6) δ 7.53(s, 6H), 6.81-6.79 (d, 2H), 6.69 (s, 1H), 3.82 (s, 6H), 3.25 (s, 3H). |
| | 2,6-dimethoxy-N-(5- | Intermediate-8 | |
| | methyl-4-oxo-6-phenyl- | | |
| | 4,5-dihydroisoxazolo[4,5- | | |
| | c]pyridin-3- | | |
| | yl)benzenesulfonamide | | |
| 5 | 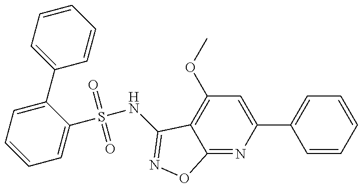 [SMILES: COc1cc(-c2ccccc2)nc2onc(NS(=O)(=O)c3ccccc3-c3ccccc3)c12] | 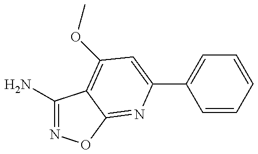 | LC-MS: 458.2 [M + H]⁺; 1H- NMR (400 MHz, DMSO-D6) δ 10.9(bs, 1H), 8.2-8.18(m, 2H), 8.14-8.12 (d, 1H), 7.62- 7.61 (m, 3H), 7.54-7.53 (m, 3H), 7.48 (s, 1H), 7.34(m, 5H), 4.04(s, 3H). |
| | N-(4-methoxy-6- | Intermediate-6 | |
| | phenylisoxazolo[5,4- | | |
| | b]pyridin-3-yl)-[1,1′- | | |
| | biphenyl]-2-sulfonamide | | |
| 6 | 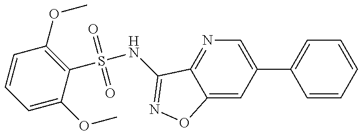 [SMILES: COc1cccc(OC)c1S(=O)(=O)Nc1noc2cc(-c3ccccc3)cnc12] | 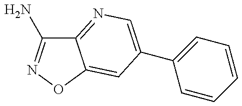 [SMILES: Nc1noc2cc(-c3ccccc3)cnc12] | LC-MS: 412.0 [M + H]⁺; 1H- NMR (400 MHz, DMSO-D6) δ 11.7(s, 1H), 9.04(s, 1H), 8.34(s, 1H), 7.88- 7.86 (d, 2H), 7.57-7.49 (m, |
| | 2,6-dimethoxy-N-(6- | Intermediate-2 | 4H), 6.76-6.74 |
| | phenylisoxazolo[4,5- | | (d, 2H), 3.72(s, 6H). |
| | b]pyridin-3- | | |
| | yl)benzenesulfonamide | | |
| 7 | 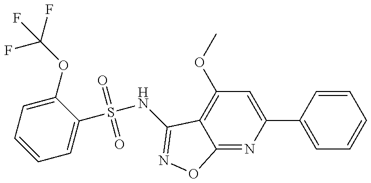 [SMILES: COc1cc(-c2ccccc2)nc2onc(NS(=O)(=O)c3ccccc3OC(F)(F)F)c12] | 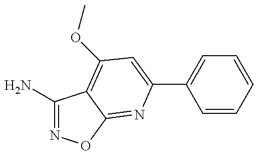 | LC-MS: 466.1 [M + H]⁺; 1H- NMR (400 MHz, DMSO-D6) δ 8.18-8.16 (m, 2H), 8.04- 8.02(d, 1H), 7.7 (bs, 1H), 7.52- 7.43 (m, 6H), 4.05 (s, 3H). |
| | N-(4-methoxy-6- | Intermediate-6 | |
| | phenylisoxazolo[5,4- | | |
| | b]pyridin-3- | | |
| | yl)-2- | | |
| | (trifluoromethoxy)benzene | | |
| | sulfonamide | | |
| 8 | 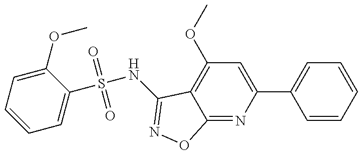 [SMILES: COc1ccccc1S(=O)(=O)Nc1noc2nc(-c3ccccc3)cc(OC)c12] | 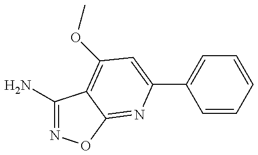 | LC-MS: 412.1 [M + H]⁺; 1H- NMR (400 MHz, DMSO-D6) δ 10.8(bs, 1H), 8.19-8.18(d, 2H), 7.8(d, 1H), 7.6 (bs, 1H), 7.53-7.50(m, 4H), 7.2(d, 1H), |
| | 2-methoxy-N-(4-methoxy- | Intermediate-6 | 7.1(t, 1H), 4.06 |
| | 6-phenylisoxazolo[5,4- | | (s, 3H), 3.80(s, |
| | b]pyridin-3- | | 3H). |
| | yl)benzenesulfonamide | | |
| 9 | 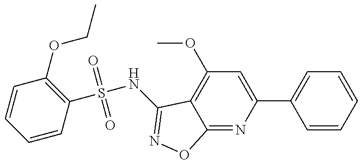 [SMILES: CCOc1ccccc1S(=O)(=O)Nc1noc2nc(-c3ccccc3)cc(OC)c12] | 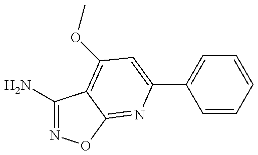 | LC-MS: 426.1 [M + H]⁺; 1H- NMR (400 MHz, DMSO-D6) δ 10.6(bs, 1H), 8.20-8.18(m, 2H), 7.8(d, 1H), 7.6 (t, 1H), 7.54-7.51(m, 3H), 7.20- 7.20(d, 1H), |
| | 2-ethoxy-N-(4-methoxy-6- | Intermediate-6 | 7.10(t, 1H), |
| | phenylisoxazolo[5,4- | | 4.11(q, 2H), 4.05 |
| | b]pyridin-3- | | (s, 3H), 1.21- |
| | yl)benzenesulfonamide | | 1.17(t, 3H). |
| 10 | 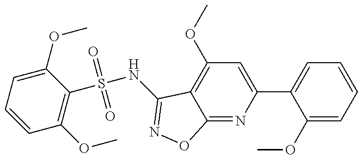 [SMILES: COc1ccccc1-c1cc(OC)c2c(NS(=O)(=O)c3c(OC)cccc3OC)noc2n1] | 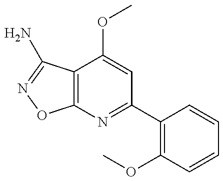 | LC-MS: 472.2 [M + H]⁺; 1H- NMR (400 MHz, DMSO-D6) δ 10.36(s, 1H), 7.74-7.72(d, 1H), 7.54-7.43 (m, 3H), 7.21- 7.18 (d, 1H), 7.10- 7.06(t, 1H), 6.81-6.79(d, 2H), 3.97(s, 3H), |
| | 2,6-dimethoxy-N-(4- | Intermediate-18 | 3.87(s, 3H), |
| | methoxy-6-(2- | | 3.79(s, 6H). |
| | methoxyphenyl)isoxazolo | | |
| | [5,4- | | |
| | b]pyridin-3- | | |
| | yl)benzenesulfonamide | | |
| 11 | 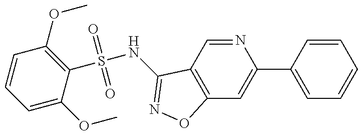 [SMILES: COc1cccc(OC)c1S(=O)(=O)Nc1noc2cc(-c3ccccc3)ncc12] | 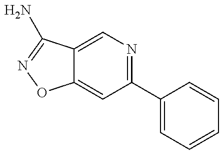 [SMILES: Nc1noc2cc(-c3ccccc3)ncc12] | LC-MS: 412.1 [M + H]⁺; 1H- NMR (400 MHz, DMSO-D6) δ 9.6(s, 1H), 8.4(bs, 1H), 8.0(d, 2H), 7.7 (s, 1H), 7.5- 7.4(m, 4H), 6.62- 6.60(d, 2H), 3.94(s, 6H). |
| | 2,6-dimethoxy-N-(6- | Intermediate-3 | |
| | phenylisoxazolo[4,5- | | |
| | c]pyridin-3- | | |
| | yl)benzenesulfonamide | | |
| 12 | 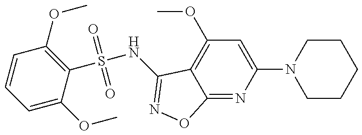 [SMILES: COc1cccc(OC)c1S(=O)(=O)Nc1noc2nc(N3CCCCC3)cc(OC)c12] | 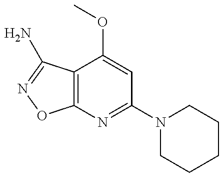 | LC-MS: 449.1 [M + H]⁺; 1H- NMR (400 MHz, DMSO-D6) δ 9.7(bs, 1H), 7.5(bs, 1H), 6.75(m, 2H), 3.88(s, 3H), 3.76(s, 3H), 3.63(bs, 3H), 3.33(m, 4H), |
| | 2,6-dimethoxy-N-(4- | Intermediate-11 | 1.61-1.52(m, |
| | methoxy-6-(piperidin-1- | | 4H), 1.24-1.23 |
| | yl)isoxazolo[5,4- | | (m, 2H). |
| | b]pyridin-3- | | |
| | yl)benzenesulfonamide | | |
| 13 | 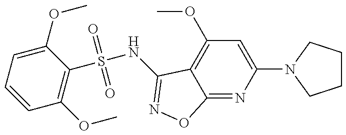 [SMILES: COc1cccc(OC)c1S(=O)(=O)Nc1noc2nc(N3CCCC3)cc(OC)c12] | 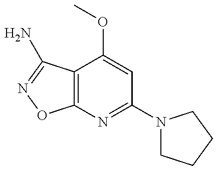 | LC-MS: 435.1 [M + H]⁺; 1H- NMR (400 MHz, DMSO-D6) δ 9.6(bs, 1H), 7.47(t, 1H), 6.772-6.751(d, 2H), 5.75(s, 1H), 3.89(s, 3H), 3.76(s, 6H), 3.44(m, 4H), 1.93(m, 4H). |
| | 2,6-dimethoxy-N-(4- | Intermediate-10 | |
| | methoxy-6-(pyrrolidin-1- | | |
| | yl)isoxazolo[5,4- | | |
| | b]pyridin-3- | | |
| | yl)benzenesulfonamide | | |
| 14 | 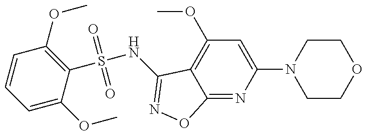 [SMILES: COc1cccc(OC)c1S(=O)(=O)Nc1noc2nc(N3CCOCC3)cc(OC)c12] | 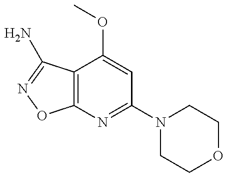 | LC-MS: 451.1 [M + H]⁺; 1H- NMR (400 MHz, DMSO-D6) δ 9.75(s, 1H), 7.47-7.43(t, 1H), 6.74-6.72(d, 2H), 6.21(s, 1H), 3.85(s, 3H), 3.73(s, 6H), 3.63-3.62(m, |
| | 2,6-dimethoxy-N-(4- | Intermediate-12 | 4H), 3.58- |
| | methoxy-6- | | 3.57(m, 4H). |
| | morpholinoisoxazolo[5,4- | | |
| | b]pyridin-3- | | |
| | yl)benzenesulfonamide | | |
| 15 | 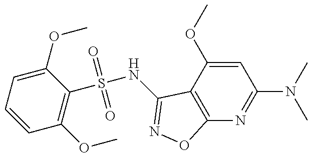 [SMILES: COc1cccc(OC)c1S(=O)(=O)Nc1noc2nc(N(C)C)cc(OC)c12] | 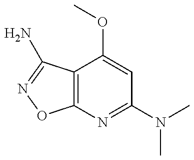 | LC-MS: 409.2 [M + H]⁺; 1H- NMR (400 MHz, DMSO-D6) δ 9.68(bs, 1H), 7.49(t, 1H), 6.78-6.76(d, 2H), 6.01(s, 1H), 3.90(s, 3H), 3.77(s, 6H), 3.10(s, 6H). |
| | N-(6-(dimethylamino)-4- | Intermediate-13 | |
| | methoxyisoxazolo[5,4- | | |
| | b]pyridin-3-yl)-2,6- | | |
| | dimethoxybenzene- | | |
| | sulfonamide | | |
| 16 | 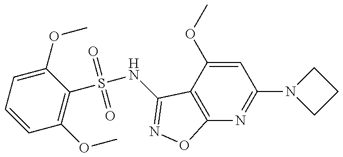 [SMILES: COc1cccc(OC)c1S(=O)(=O)Nc1noc2nc(N3CCC3)cc(OC)c12] | 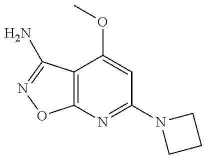 | LC-MS: 421.2 [M + H]⁺; 1H- NMR (400 MHz, DMSO-D6) δ 9.64(s, 1H), 7.48(t, 1H), 6.77-6.75(d, 2H), 5.69(s, 1H), 4.04-4.01(t, 4H), 3.85(s, 3H), 3.75(s, 6H), 2.34- 2.30(m, 2H). |
| | N-(6-(azetidin-1-yl)-4- | Intermediate-14 | |
| | methoxyisoxazolo[5,4- | | |
| | b]pyridin-3-yl)-2,6- | | |
| | dimethoxybenzene- | | |
| | sulfonamide | | |
| 17 | 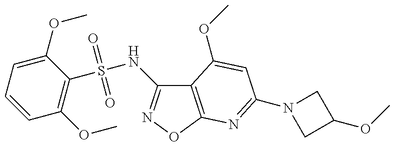 [SMILES: COc1cccc(OC)c1S(=O)(=O)Nc1noc2nc(N3CC(OC)C3)cc(OC)c12] | 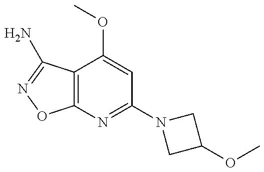 | LC-MS: 451.0 [M + H]⁺; 1H- NMR (400 MHz, DMSO-D6) δ 9.69(s, 1H), 7.50-7.45(t, 1H), 6.77-6.74 (d, 2H), 5.76(s, 1H), 4.31-4.30 (m, 1H), 4.23- 4.19(m, 2H), |
| | 2,6-dimethoxy-N-(4- | Intermediate-15 | 3.86-3.82(m, |
| | methoxy-6-(3- | | 5H), 3.75(s, 6H), |
| | methoxyazetidin-1- | | 3.23(s, 3H). |
| | yl)isoxazolo[5,4- | | |
| | b]pyridin-3- | | |
| | yl)benzenesulfonamide | | |
| 18 | 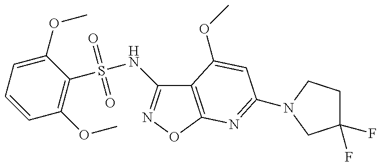 [SMILES: COc1cccc(OC)c1S(=O)(=O)Nc1noc2nc(N3CCC(F)(F)C3)cc(OC)c12] | 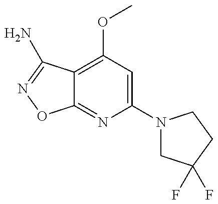 | LC-MS: 471.0 [M + H]⁺; 1H- NMR (400 MHz, DMSO-D6) δ 9.75(bs, 1H), 7.48(t, 1H), 6.77-6.75(d, 2H), 5.9(s, 1H), 3.94-3.88(m, 5H), 3.75(s, 6H), 3.71-3.68(t, 2H), 2.56(m, 2H). |
| | N-(6-(3,3- | Intermediate-16 | |
| | difluoropyrrolidin-1-yl)-4- | | |
| | methoxyisoxazolo[5,4- | | |
| | b]pyridin-3-yl)-2,6- | | |
| | dimethoxybenzene- | | |
| | sulfonamide | | |
| 19 | 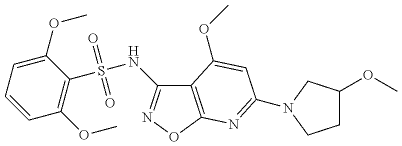 [SMILES: COc1cccc(OC)c1S(=O)(=O)Nc1noc2nc(N3CCC(OC)C3)cc(OC)c12] | 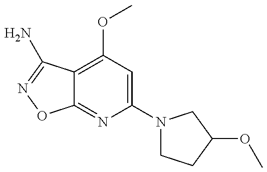 | LC-MS: 465.2 [M + H]⁺; 1H- NMR (400 MHz, DMSO-D6) δ 9.62(bs, 1H), 7.49-7.45(t, 1H), 6.77-6.74 (d, 2H), 5.9(s, 1H), 4.09-4.05 (m, 1H), 3.89(s, 3H), 3.75(s, 6H), |
| | 2,6-dimethoxy-N-(4- | Intermediate-17 | 3.55-3.52(m, |
| | methoxy-6-(3- | | 2H) 3.44-3.37(m, |
| | methoxypyrrolidin-1- | | 2H), 3.25(s, |
| | yl)isoxazolo[5,4-b]pyridin- | | 3H), 2.05(m, 2H). |
| | 3-yl)benzenesulfonamide | | |
| 20 | 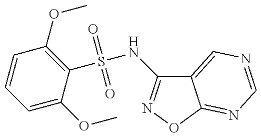 [SMILES: COc1cccc(OC)c1S(=O)(=O)Nc1noc2ncncc12] | 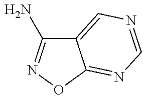 | LC-MS: 337.1 [M + H]⁺; 1H- NMR (400 MHz, DMSO-D6) δ 12.2(bs, 1H), 9.5(s, 1H), 9.2(s, 1H), 7.5 (t, 1H), 6.77- 6.75(d, 2H), 3.75(s, 6H). |
| | N-(isoxazolo[5,4- | | |
| | d]pyrimidin-3-yl)- | | |
| | 2,6- | | |
| | dimethoxybenzene- | | |
| | sulfonamide | | |
| 21 | 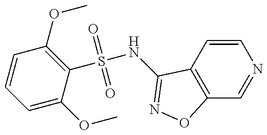 [SMILES: COc1cccc(OC)c1S(=O)(=O)Nc1noc2cnccc12] | 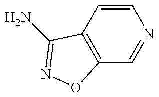 | LC-MS: [M + H]⁺; 1H-NMR (400 MHz, DMSO-D6) δ 11.8(s, 1H), 9.09 (s, 1H), 8.55- 8.54(d, 1H), 8.1- 8.08(d, 1H), 7.50-7.46(t, 1H), |
| | N-(isoxazolo[5,4- | | 6.75-6.73(d, |
| | c]pyridin-3-yl)- | | 2H), 3.72(s, 6H). |
| | 2,6- | | |
| | dimethoxybenzene- | | |
| | sulfonamide | | |
| 22 | 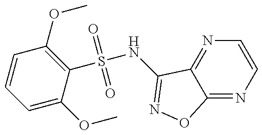 [SMILES: COc1cccc(OC)c1S(=O)(=O)Nc1noc2nccnc12] | 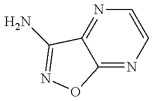 | LC-MS: [M + H]⁺; 1H-NMR (400 MHz, DMSO-D6) δ 12.08(bs, 1H), 8.88(s, 1H), 8.74(s, 1H), 7.51- 7.47(t, 1H), 6.77-6.75(d, 2H), 3.72(s, 6H). |
| | N-(isoxazolo[4,5- | | |
| | b]pyrazin-3-yl)- | | |
| | 2,6- | | |
| | dimethoxybenzene- | | |
| | sulfonamide | | |
| 23 | 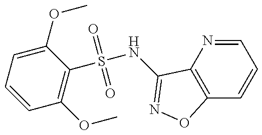 [SMILES: COc1cccc(OC)c1S(=O)(=O)Nc1noc2cccnc12] | 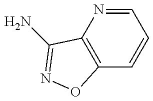 | LC-MS: [M + H]⁺; 1H-NMR (400 MHz, DMSO-D6) δ 8.51-8.50(d, 1H), 7.85-7.83(d, 1H), 7.46-7.43 (m, 1H), 7.3- 7.26(t, 1H), 6.64- |
| | N-(isoxazolo[4,5- | | 6.62(d, 2H), |
| | b]pyridin-3-yl)- | | 3.59(s, 6H). |
| | 2,6- | | |
| | dimethoxybenzene- | | |
| | sulfonamide | | |
| 24 | 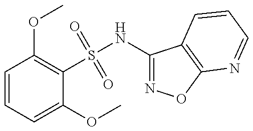 [SMILES: COc1cccc(OC)c1S(=O)(=O)Nc1noc2ncccc12] | 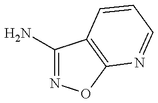 | LC-MS: [M + H]⁺; 1H-NMR (400 MHz, DMSO-D6) δ 11.72(s, 1H), 8.61-8.60(d, 1H), 8.54-8.51 (dd, 1H), 7.50- 7.43(m, 2H), |
| | N-(isoxazolo[5,4- | | 6.74-6.71(d, |
| | b]pyridin-3-yl)- | | 2H), 3.71(s, 6H). |
| | 2,6- | | |
| | dimethoxybenzene- | | |
| | sulfonamide | | |
| 25 | 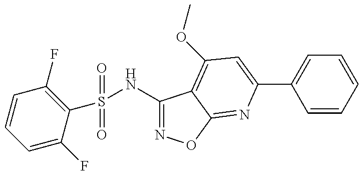 [SMILES: COc1cc(-c2ccccc2)nc2onc(NS(=O)(=O)c3c(F)cccc3F)c12] | 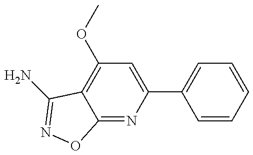 | LC-MS: 418.2 [M + H]⁺; 1H- NMR (400 MHz, DMSO-D6) δ 8.18-8.16 (m, 2H), 7.64 (brs, 1H), 7.58- 7.48 (m, 3H), 7.44 (s, 1H), 7.25-7.15 (m, 2H), 4.05 (s, 3H). |
| | 2,6-difluoro-N-(4- | Intermediate-6 | |
| | methoxy-6- | | |
| | phenylisoxazolo[5,4- | | |
| | b]pyridin-3- | | |
| | yl)benzenesulfonamide | | |
| 26 | 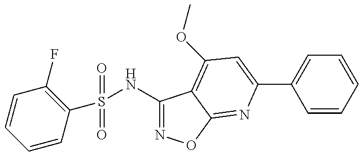 [SMILES: COc1cc(-c2ccccc2)nc2onc(NS(=O)(=O)c3ccccc3F)c12] | 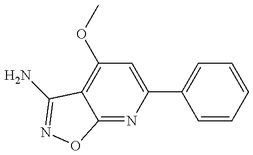 | LC-MS: 400.2 [M + H]⁺; 1H- NMR (400 MHz, DMSO-D6) δ 8.16-8.13 (m, 2H), 7.86 (t, 1H), 7.73-7.64 (m, 2H), 7.50- 7.40 (m, 4H), 7.39-7.34 (m, 2H), 4.02 (s, 3H). |
| | 2-fluoro-N-(4-methoxy-6- | Intermediate-6 | |
| | phenylisoxazolo[5,4- | | |
| | b]pyridin-3- | | |
| | yl)benzenesulfonamide | | |
| 27 | 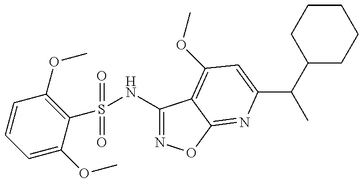 [SMILES: COc1cccc(OC)c1S(=O)(=O)Nc1noc2nc(C(C)C3CCCCC3)cc(OC)c12] | 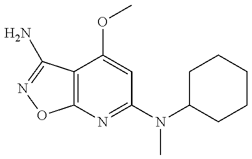 | LC-MS: 477.2 [M + H]⁺; 1H- NMR (400 MHz, DMSO-D6) δ 9.65 (brs, 1H), 7.47 (t, 1H), 6.76 (d, 2H), 4.45 (brs, 1H), 3.90 (s, 3H), 3.76 (s, 6H), 2.91 (s, 3H), 1.81- 1.75 (m, 2H), |
| | N-(6- | Intermediate-18 | 1.68-1.29 (m, |
| | (cyclohexyl(methyl)amino)- | | 6H), 1.05- |
| | 4-methoxyisoxazolo[5,4- | | 1.25 (m, 2H). |
| | b]pyridin-3-yl)-2,6- | | |
| | dimethoxybenzene- | | |
| | sulfonamide | | |
| 28 | 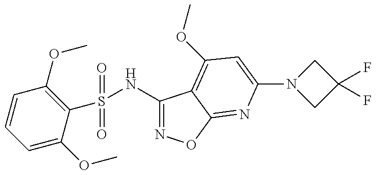 [SMILES: COc1cccc(OC)c1S(=O)(=O)Nc1noc2nc(N3CC(F)(F)C3)cc(OC)c12] | 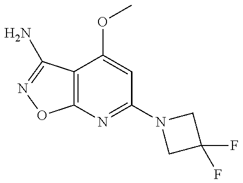 | LC-MS: 457.2 [M + H]⁺; 1H- NMR (400 MHz, CDCl₃) δ 7.88 (brs, 1H), 7.40 (t, 1H), 6.60 (d, 2H), 5.54 (s, 1H), 4.44 (t, 4H), 4.02 (s, 3H), 3.89 (s, 6H). |
| | N-(6-(3,3-difluoroazetidin- | Intermediate-19 | |
| | 1-yl)-4- | | |
| | methoxyisoxazolo[5,4- | | |
| | b]pyridin-3-yl)-2,6- | | |
| | dimethoxybenzene- | | |
| | sulfonamide | | |
| 29 | 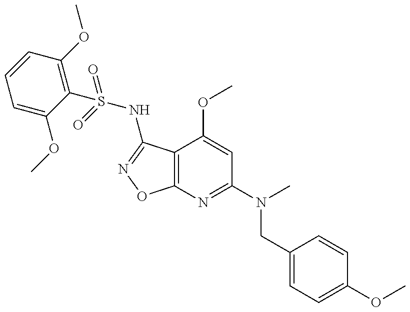 [SMILES: COc1ccc(CN(C)c2cc(OC)c3c(NS(=O)(=O)c4c(OC)cccc4OC)noc3n2)cc1] | 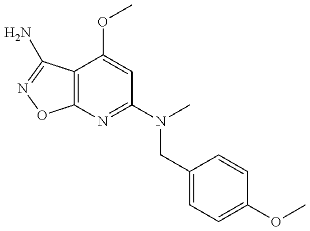 | LC-MS: 514.95 [M + H]⁺; 1H- NMR (400 MHz, DMSO-D6) δ 9.66 (brs, 1H), 7.24 (brs, 1H), 7.12 (d, 2H), 6.82 (d, 2H), 6.61 (brs, 2H), 5.88 (brs, 1H), 4.69 (s, 2H), 3.83 (s, 3H), 3.67 (s, 3H), 3.62 (brs, 6H) 2.98 (s, 3H). |
| | 2,6-dimethoxy-N-(4- | Intermediate-20 | |
| | methoxy-6-((4- | | |
| | methoxybenzyl)(methyl) | | |
| | amino)isoxazolo[5,4- | | |
| | b]pyridin-3- | | |
| | yl)benzenesulfonamide | | |
| 30 | 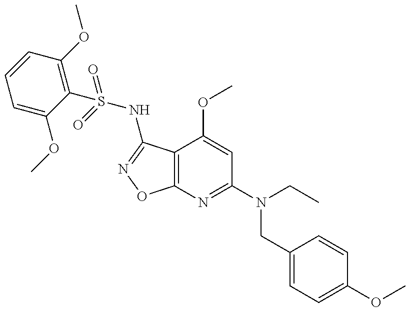 [SMILES: CCN(Cc1ccc(OC)cc1)c1cc(OC)c2c(NS(=O)(=O)c3c(OC)cccc3OC)noc2n1] | 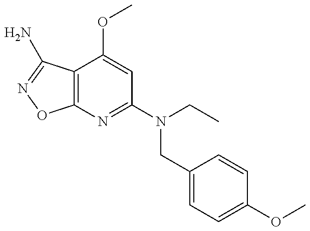 | LC-MS: 529.3 [M + H]⁺; 1H- NMR (400 MHz, DMSO-D6) δ 9.74 (brs, 1H), 7.53 (t, 1H), 7.22 (d, 2H), 6.91 (d, 2H), 6.81 (d, 2H), 6.06 (s, 1H), 4.76 (s, 2H), 3.87 (s, 3H) 3.82 (s, 6H), 3.75 (s, 3H), 3.61- 3.55 (m, 2H), 1.14 (t, 3H). |
| | N-(6-(ethyl(4- | Intermediate-20 | |
| | methoxybenzyl)amino)-4- | | |
| | methoxyisoxazolo[5,4- | | |
| | b]pyridin-3-yl)-2,6- | | |
| | dimethoxybenzene- | | |
| | sulfonamide | | |
| 31 | 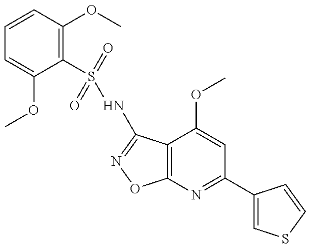 [SMILES: COc1cccc(OC)c1S(=O)(=O)Nc1noc2nc(-c3ccsc3)cc(OC)c12] | 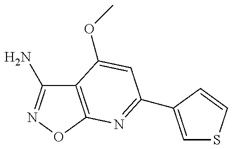 | LC-MS: 448.2 [M + H]⁺; 1H- NMR (400 MHz, DMSO-D6) δ 10.35 (s, 1H), 8.52-8.50 (m, 1H), 7.91 (d, 1H), 7.76- 7.72 (m, 1H), 7.56 (t, 1H), 7.50 (s, 1H), 6.84 (d, 2H), 4.09 (s, 3H), 3.82 (s, 6H). |
| | 2,6-dimethoxy-N-(4- | Intermediate-23 | |
| | methoxy-6-(thiophen-3- | | |
| | yl)isoxazolo[5,4- | | |
| | b]pyridin-3- | | |
| | yl)benzenesulfonamide | | |
| 32 | 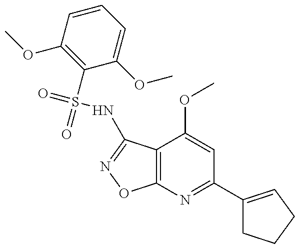 [SMILES: COc1cccc(OC)c1S(=O)(=O)Nc1noc2nc(C3=CCCC3)cc(OC)c12] | 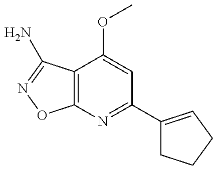 | LC-MS: 432.2 [M + H]⁺; 1H- NMR (400 MHz, DMSO-D6) δ 10.21 (s, 1H), 7.48 (t, 1H), 7.10 (s, 1H), 6.88 (s, 1H), 6.75 (d, 2H), 3.95 (s, 3H), 3.73 (s, 6H), 2.72 (br t, 2H) 2.59- 2.49 (m, 2H), 1.99-1.92 (m, 2H). |
| | N-(6-(cyclopent-1-en-1- | Intermediate-24 | |
| | yl)-4- | | |
| | methoxyisoxazolo[5,4- | | |
| | b]pyridin-3-yl)-2,6- | | |
| | dimethoxybenzene- | | |
| | sulfonamide | | |
| 33 | 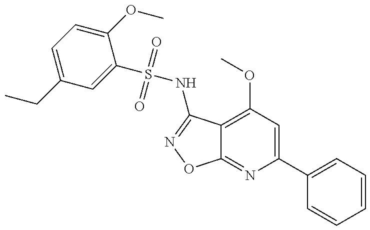 [SMILES: CCc1ccc(OC)c(S(=O)(=O)Nc2noc3nc(-c4ccccc4)cc(OC)c23)c1] | 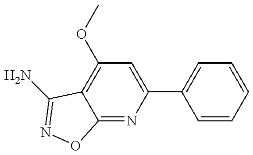 | LC-MS: 440.1 [M + H]⁺; 1H- NMR (400 MHz, DMSO-D6) δ 10.74 (brs, 1H), 8.22-8.17 (m, 2 H), 7.64 (d, 1H), 7.55- 7.45 (m, 5H), 7.13 (d, 1H), 4.06 (s, 3H), 3.76 (s, 3H), 2.62 (q, 2H), 1.16 (t, 3H). |
| | 5-ethyl-2-methoxy-N-(4- | Intermediate-6 | |
| | methoxy-6- | | |
| | phenylisoxazolo[5,4- | | |
| | b]pyridin-3- | | |
| | yl)benzenesulfonamide | | |
| 34 | 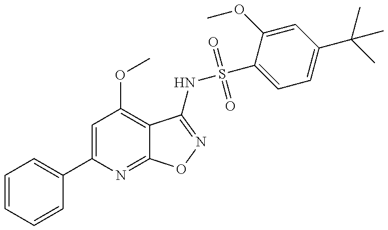 [SMILES: COc1cc(C(C)(C)C)ccc1S(=O)(=O)Nc1noc2nc(-c3ccccc3)cc(OC)c12] | 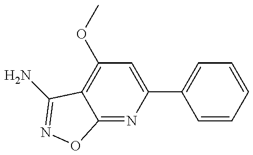 | LC-MS: 468.1 [M + H]⁺; 1H- NMR (400 MHz, DMSO-D6) δ 10.71 (s, 1H), 8.21-8.18 (m, 2H), 7.75 (d, 1H), 7.56-7.51 (m, 4H), 7.16- 7.13 (m, 2H), 4.06 (s, 3H), 3.83 (s, 3H), 1.32(s, 9H). |
| | 4-(tert-butyl)-2-methoxy- | Intermediate-6 | |
| | N-(4-methoxy-6- | | |
| | phenylisoxazolo[5,4- | | |
| | b]pyridin-3- | | |
| | yl)benzenesulfonamide | | |

### Example-2: N-(6-((1H-pyrazol-1-yl)methyl)-4-methoxyisoxazolo[5,4-b]pyridin-3-yl)-2,6-dimethoxybenzenesulfonamide (Compound-35)

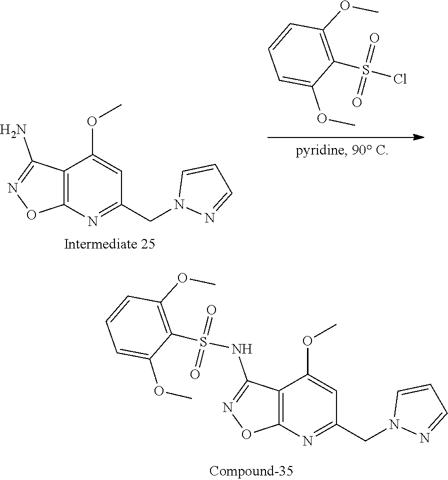 [SMILES: COc1cc(Cn2cccn2)nc2onc(N)c12 ; COc1cccc(OC)c1S(=O)(=O)Cl ; COc1cccc(OC)c1S(=O)(=O)Nc1noc2nc(Cn3cccn3)cc(OC)c12]

To a mixture of 6-((1H-pyrazol-1-yl)methyl)-4-methoxyisoxazolo[5,4-b]pyridin-3-amine (0.060 g, 0.245 mmol, 1 equiv.) and 2,6-dimethoxybenzenesulfonyl chloride (0.174 g, 0.735 mmol, 3 equiv.) in sealed tube was added pyridine (0.5 mL) at RT. Then the reaction mixture was stirred at 90° C. for 16 h. After completion of reaction, the reaction mixture was concentrated under vacuum to get the crude material. The crude was purified by Prep TLC using 5% Methanol and dichloromethane. The solid was washed with pentane (3 mL) to get the pure compound of N-(6-((1H-pyrazol-1-yl)methyl)-4-methoxyisoxazolo[5,4-b]pyridin-3-yl)-2,6-dimethoxybenzenesulfonamide (0.006 g, 5.50%). LC-MS: 446.2 [M+H]⁺; 1H-NMR (400 MHz, DMSO-D6) δ 10.42 (brs, 1H), 7.93 (s, 1H), 7.56-7.50 (m, 2H), 6.85-6.79 (m, 3H), 6.35 (s, 1H), 5.53 (s, 2H), 3.91 (s, 3H), 3.79 (s, 6H).

### Example-3: Preparation of 2,6-dimethoxy-N-(4-methoxy-6-phenylisoxazolo[5,4-b]pyridin-3-yl)-N-methylbenzenesulfonamide (Compound-36)

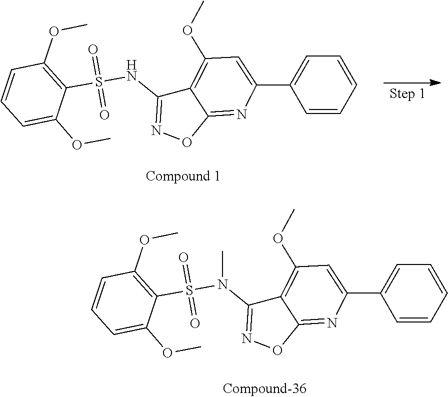 [SMILES: COc1cccc(OC)c1S(=O)(=O)Nc1noc2nc(-c3ccccc3)cc(OC)c12 ; COc1cccc(OC)c1S(=O)(=O)N(C)c1noc2nc(-c3ccccc3)cc(OC)c12]

To a solution of 2,6-dimethoxy-N-(4-methoxy-6-phenylisoxazolo[5,4-b]pyridin-3-yl)benzenesulfonamide (Compound 1, 0.100 g, 0.227 mmol) in THF (2 mL) was added sodium hydride (60%) (0.008 g, 0.34 mmol) at 0° C. and stirred the reaction mixture at RT for 30 min. The reaction mixture was cooled at 0° C. and methyl iodide (0.064 g, 0.450 mmol) was added. Then the reaction mixture was stirred at RT for 12 h. The reaction mixture was then quenched with NH₄Cl and extracted with ethyl acetate. The combined organic layers were washed with water and brine, dried over sodium sulphate, concentrated, and purified by preparative TLC using 2% methanol in DCM as eluent to give pure title compound (0.004 g, 3.87%). LC-MS: 456.3 [M+H]⁺; 1H-NMR (400 MHz, DMSO-D6) δ 8.24-8.21 (m, 2H), 7.60-7.54 (m, 5H), 6.81 (d, 2H), 4.02 (s, 3H), 3.68 (s, 6H), 3.36 (s, 3H).

### Example-4: 2,6-dimethoxy-N-(4-methoxy-6-(methylamino)isoxazolo[5,4-b]pyridin-3-yl)benzenesulfonamide (Compound-37)

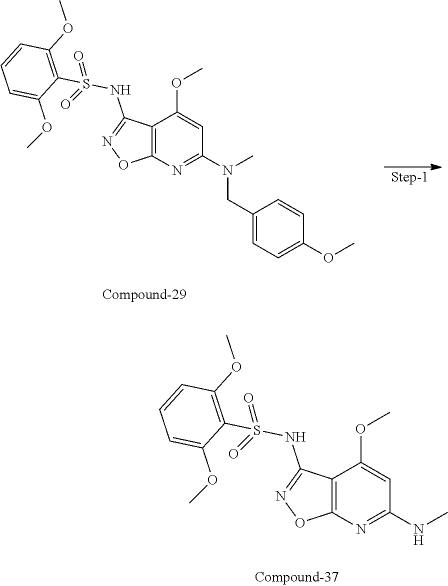 [SMILES: COc1ccc(CN(C)c2cc(OC)c3c(NS(=O)(=O)c4c(OC)cccc4OC)noc3n2)cc1 ; CNc1cc(OC)c2c(NS(=O)(=O)c3c(OC)cccc3OC)noc2n1]

To a solution of 2,6-dimethoxy-N-(4-methoxy-6-((4-methoxybenzyl)(methyl) amino)isoxazolo[5,4-b]pyridin-3-yl)benzenesulfonamide (0.06 g, 0.117 mmol) in DCM (3 mL) was added 4M HCl in dioxane (0.29 mL, 1.17 mmol) and stirred at RT for 4 h. After the completion of reaction, the reaction mixture was concentrated and triturated with diethyl ether and purified by preparative TLC using 2% methanol in DCM as eluent to give pure title compound (0.005 g, 10.84%). LC-MS: 395.0 [M+H]⁺; 1H-NMR (400 MHz, DMSO-D6) δ 9.54 (brs, 1H), 7.45 (t, 1H), 7.40 (brs, 1H), 6.73 (d, 2H), 5.85 (s, 1H), 3.77 (s, 3H), 3.73 (s, 6H), 2.76 (d, 3H).

### Example-5: N-(6-(ethylamino)-4-methoxyisoxazolo[5,4-b]pyridin-3-yl)-2,6-dimethoxybenzenesulfonamide (Compound-38)

The title compound (Example-38) was prepared according to the procedure described in the synthesis of Example-4 for compound 37 with appropriate variations in reactants, quantities of reagents and solvents.

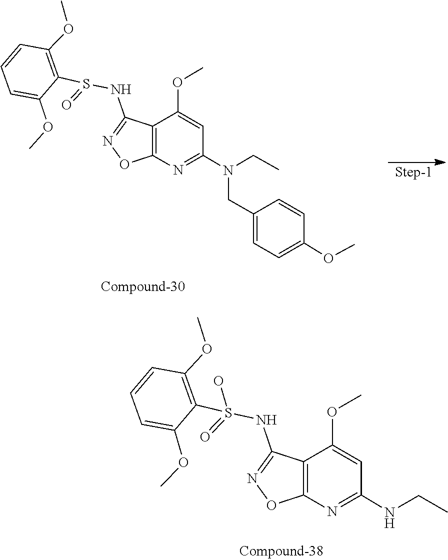

LC-MS: 409.2 [M+H]⁺; 1H-NMR (400 MHz, DMSO-D6) δ 7.29 (t, 1H), 7.12 (brs, 1H), 6.65 (d, 2H), 5.75 (s, 1H), 3.80 (s, 3H), 3.67 (s, 6H), 3.26-3.20 (m, 2H), 1.10 (t, 3H).

### Example-6: KAT6A Biochemical Assay

A TR-FRET based method was used for assaying compounds of the invention for KAT6A enzyme inhibitory activity. TR-FRET is homogeneous proximity assay where Europium-labelled anti-acetyl lysine antibody binds to the acetylated substrate labelled with biotin which in turn binds to streptavidin labelled APC fluorescence acceptor. Europium can transfer energy to APC in the complex and the interaction of two dye-labelled binding partners is detected by the energy transfer between a donor and an acceptor dye, and the subsequent light emission by the acceptor dye. KAT6A transfer an acetyl group from acetyl CoA to lysine amino acids of histones/target proteins. Typically, 5 μL of human-KAT6A (MYST domain 507-778 aa) in assay buffer (100 mM Tris HCl (pH 7.8), 15 mM NaCl, 1 mM EDTA, 0.01% Tween-20, 0.02% BSA, 1 mM DTT) is added to a 384-well plate containing 5 μL of selected test compound in final 1% DMSO, serially diluted in 1:3 in an 8-10-point titration. The selected compound of the present invention and enzyme are incubated for 30 min at room temperature. Next, 5 μL of substrate mix containing histone H4 peptide and acetyl-CoA in assay buffer is added to the plate. The final concentrations of H4 peptide and acetyl-CoA are 200 nM and 600 nM respectively. Following 30 min reaction at room temperature, 5 μL of detection mix containing Europium labelled anti-acetyl antibody and streptavidin-APC is added to the reaction wells. Plate is further incubated for 45 min at room temperature and is read in TR-FRET mode (Ex: 340 nm; Em: 615 nm and 665 nm) on a plate reader. The activity of the test compound on inhibition of KAT6A is expressed as percent inhibition of internal assay controls as presented in Table-1 below.

TABLE 1
Percent inhibition data in KAT6A

| | Percent Inhibition |
|---|---|
| Compound | (at 1 μM) |
| 1 | 100 |
| 2 | 100 |
| 3 | 96 |
| 4 | 23 |
| 5 | 52 |
| 6 | 58 (at 10 μM) |
| 7 | 25 |
| 8 | 82 |
| 9 | 62 |
| 10 | 100 |
| 11 | 12 |
| 12 | 54 |
| 13 | 97 |
| 14 | 49 |
| 15 | 59 |
| 16 | 100 |
| 17 | 100 |
| 18 | 100 |
| 19 | 92 |
| 20 | 14 |
| 21 | 11 |
| 22 | 12 |
| 24 | 13 |
| 25 | 30 |
| 26 | 23 |
| 27 | 17 |
| 28 | 80 |
| 29 | 38 |
| 30 | 56 |
| 34 | 100 (at 10 μm) |
| 36 | 0 |
| 37 | 34 |
| 38 | 95 |

Selected compounds of the present invention were screened in the above-mentioned assay procedure and IC₅₀ values were determined by fitting the dose-response data to sigmoidal curve fitting equation using Graph pad prism software V8. The results are summarized into groups A, B and C in the table below. Herein group “A” encompasses the compounds having IC₅₀ values lower than 0.1 μM, “B” encompasses the compounds having IC₅₀ values between 0.11 μM and 0.2 μM (both inclusive) and “C” encompasses the compounds having IC₅₀ values higher than 0.2 μM. The results are given below.

TABLE 2
IC₅₀ values for the compounds of this invention

| Compound | IC₅₀ (μM) |
|---|---|
| 1 | A |
| 2 | A |
| 3 | B |
| 5 | A |
| 8 | B |
| 9 | C |
| 10 | A |
| 13 | B |
| 15 | C |
| 16 | B |
| 17 | B |
| 18 | A |
| 19 | B |
| 28 | C |
| 31 | A |
| 32 | B |
| 33 | A |
| 34 | C |
| 35 | C |

### INCORPORATION BY REFERENCE

All publications and patents mentioned herein are hereby incorporated by reference in their entirety as if each individual publication or patent were specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of formula (I): 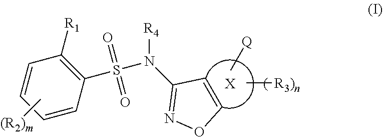
or a pharmaceutically acceptable salt or a stereoisomer or a tautomer thereof; wherein,
R₁ represents hydrogen, halogen, alkyl, alkoxy, haloalkoxy or aryl; wherein the alkyl is unsubstituted or substituted with one or more substituents selected from hydroxy, halogen, amino and amido; and the aryl is unsubstituted or substituted with one or more substituents selected from alkyl, alkoxy, hydroxy, cyano, halogen and amino;
R₂, at each occurrence, represents hydrogen, halogen, alkyl or alkoxy; wherein the alkyl and alkoxy are unsubstituted or substituted with one or more substituents selected from hydroxy, halogen, amino and amido;
X represents 6-membered fused heteroaryl ring or 6-membered fused heterocycloalkyl ring;
R₃, at each occurrence, represents hydrogen, alkyl, alkoxy or heteroaralkyl; wherein the alkyl and alkoxy groups are unsubstituted or substituted with one or more substituents selected from hydroxy, amino, halogen and amido; or
any two R₃ groups attached to the same carbon atom combine together to form an oxo group;
R₄ represents hydrogen or alkyl; wherein the alkyl is unsubstituted or substituted with one or more hydroxy or halogen groups;
Q represents hydrogen, —NRₐR_{b}, alkyl, aryl, 4- to 6-membered heterocycloalkyl, 3- to 8-membered cycloalkenyl or 5- to 6-membered heteroaryl; wherein the aryl, heterocycloalkyl, cycloalkenyl and heteroaryl are unsubstituted or substituted with one or more substituents selected from alkyl, alkoxy, amino, amido, halogen, haloalkyl, hydroxyalkyl and oxo; and the alkyl is unsubstituted or substituted with 5- to 6-membered heteroaryl;
Rₐ and R_{b} are independently selected from hydrogen, alkyl, 5- to 6-membered cycloalkyl, aryl and aryl-alkyl; wherein the cycloalkyl, aryl, and aryl-alkyl are unsubstituted or substituted with one or more substituents selected from alkyl, alkoxy, amino, halogen, haloalkyl and hydroxyalkyl;
m represents 1, 2 or 3; and
n represents 1, 2 or 3.

2. The compound of claim 1, wherein X represents 6-membered fused heteroaryl ring containing 1, 2 or 3 N atoms.

3. The compound of claim 1 or 2, wherein X represents 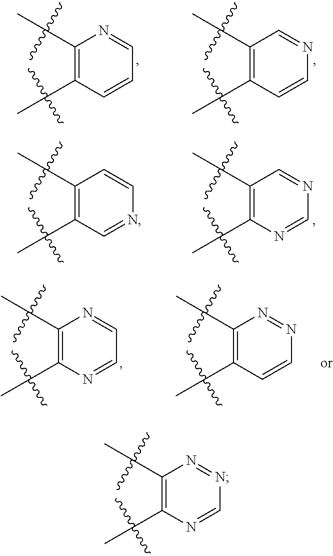
wherein [symbol] represents the points of fusion with isoxazolyl ring of formula (I).

4. The compound of any one of claims 1 to 3, wherein Q represents hydrogen, —NRₐR_{b}, phenyl, 4- to 6-membered heterocycloalkyl, 3- to 8-membered cycloalkenyl, 5- to 6-membered heteroaryl or an alkyl substituted with 5- to 6-membered heteroaryl.

5. The compound of any one of claims 1 to 4, wherein Q represents
(i) hydrogen, —NH(CH₃), —N(CH₃)(CH₃), —N(CH₂CH₃)(CH₂CH₃), —NH(CH₂CH₃), —N(CH₃)(CH₂CH₃), —N(CH₃)(phenyl), —N(CH₃)(cyclohexyl), —N(CH₂CH₃)(phenyl), —N(CH₂CH₃)(CH₂-phenyl), —N(CH₃)(CH₂-phenyl), —N(CH₂CH₃)(cyclohexyl) or —CH₂-pyrazolyl; wherein the phenyl, cyclohexyl, and pyrazolyl are unsubstituted or substituted with 1 or 2 substituents selected from alkyl, alkoxy, halogen, haloalkyl and hydroxyalkyl; or
(ii) phenyl, azetidinyl, thietanyl, oxetanyl, oxazetidinyl, pyrrolidinyl, tetrahydrofuranyl, tetrahydrothiophenyl, imidazolidinyl, oxazolidinyl, thiazolidinyl, piperidinyl, tetrahydropyranyl, morpholinyl, dioxanyl, cyclopentenyl, cyclohexenyl, furanyl, thiophenyl, pyrrolyl, oxazolyl, imdazolyl, thiazolyl, isothiazolyl, pyrazolyl, isoxazolyl, pyridinyl, pyranyl, pyridazinyl, pyrimidinyl or pyrazinyl; wherein each group is unsubstituted or substituted with 1 or 2 substituents selected from alkyl, alkoxy, halogen, haloalkyl and hydroxyalkyl.

6. The compound of any one of claims 1 to 5, wherein R₃ represents hydrogen, (C₁-C₄)alkyl or (C₁-C₄)alkoxy; or any two R₃ groups attached to the same carbon atom combine together to form an oxo group.

7. The compound of claim 1, represented by compound of formula (IA): 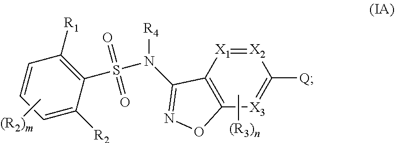
wherein, [symbol]
represents a single bond or double bond; and
X₁, X₂ and X₃ independently represents N or C; wherein at least one of X₁, X₂ and X₃ is N.

8. The compound of claim 7, wherein
R₁ represents hydrogen, halogen, (C₁-C₄)alkyl, (C₁-C₄)alkoxy, halo-(C₁-C₄)alkoxy or aryl;
R₂, at each occurrence, represents hydrogen, halogen, (C₁-C₄)alkyl or (C₁-C₄)alkoxy;
R₃, at each occurrence, represents hydrogen, (C₁-C₄)alkyl or (C₁-C₄)alkoxy; or any two R₃ groups attached to the same carbon atom combine together to form an oxo group;
R₄ represents hydrogen or (C₁-C₄)alkyl; and
Q represents,
(i) hydrogen, —NH(CH₃), —N(CH₃)(CH₃), —NH(CH₂CH₃), —N(CH₃)(phenyl), —N(CH₃)(cyclohexyl), —N(CH₂CH₃)(phenyl), —N(CH₂CH₃)(CH₂-phenyl), —N(CH₃)(CH₂-phenyl), —N(CH₂CH₃)(cyclohexyl) or —CH₂-pyrazolyl; wherein the phenyl, cyclohexyl, and pyrazolyl are unsubstituted or substituted with 1 or 2 substituents selected from alkyl, alkoxy, halogen, haloalkyl and hydroxyalkyl; or
(ii) phenyl, azetidinyl, oxetanyl, pyrrolidinyl, tetrahydrofuranyl, imidazolidinyl, oxazolidinyl, thiazolidinyl, piperidinyl, tetrahydropyranyl, morpholinyl, cyclopentenyl, cyclohexenyl, furanyl, thiophenyl, pyrrolyl, isothiazolyl, pyrazolyl, isoxazolyl, pyridinyl, pyranyl, pyridazinyl, pyrimidinyl or pyrazinyl; wherein each group is unsubstituted or substituted with 1 or 2 substituents selected from alkyl, alkoxy, halogen and haloalkyl;
m represents 1 or 2; and
n represents 1, 2 or 3.

9. The compound of claim 1, represented by compound of formula (IB): 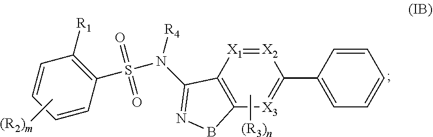
wherein,
X₁, X₂ and X₃ independently represents N or C; wherein at least one of X₁, X₂ and X₃ is N.

10. The compound of claim 9, wherein,
R₁ represents hydrogen, —Cl, —F, —O—CH₃, —O—CH₂CH₃, —O—CF₃ or phenyl;
R₂, at each occurrence, represents —Cl, —F, —CH₃, —CH₂CH₃, —C(CH₃)₃, —O—CH₃ or —O—CH₂CH₃;
R₃, at each occurrence, represents hydrogen, —CH₃, —CH₂CH₃, —C(CH₃)₃, —O—CH₃, —O—CH₂CH₃; or any two R₃ groups attached to the same carbon atom combine together to form an oxo group;
m represents 1 or 2; and
n represents 1, 2 or 3.

11. The compound of claim 1, represented by compound of formula (IC): 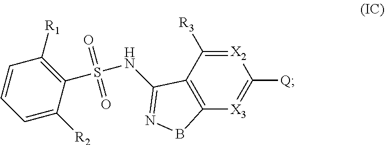
wherein,
X₂ and X₃ independently represents N or C; wherein at least one of X₂ and X₃ is N; and
Q represents 4- to 6-membered heterocycloalkyl, 3- to 8-membered cycloalkenyl or 5- to 6-membered heteroaryl.

12. The compound of claim 11, wherein Q represents 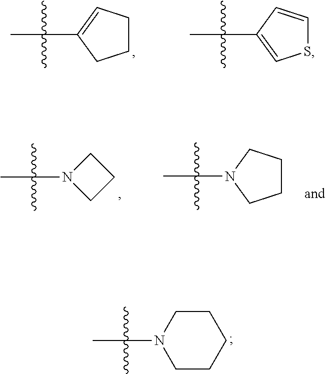 [SMILES: *C1=CCCC1 ; *c1ccsc1 ; *N1CCC1 ; *N1CCCC1 ; *N1CCCCC1]
wherein each group is unsubstituted or substituted with one or two occurrences of —Cl, —F, —OCH₃ or —OCH₂CH₃.

13. The compound of claim 11, wherein,
R₁ represents hydrogen, —Cl, —F, —O—CH₃, —O—CH₂CH₃, —O—CF₃ or phenyl;
R₂, at each occurrence, represents hydrogen, —Cl, —F, —CH₃, —CH₂CH₃, —C(CH₃)₃, —O—CH₃ or —O—CH₂CH₃;
R₃, at each occurrence, represents hydrogen, —CH₃, —CH₂CH₃, —C(CH₃)₃, —O—CH₃, —O—CH₂CH₃; or any two R₃ groups attached to the same carbon atom to form an oxo group; and
Q represents 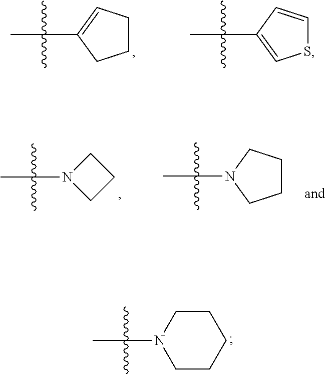 [SMILES: *C1=CCCC1 ; *c1ccsc1 ; *N1CCC1 ; *N1CCCC1 ; *N1CCCCC1]
wherein each group is unsubstituted or substituted with one or two occurrences of —Cl, —F, —OCH₃ or —OCH₂CH₃.

14. The compound of any one of claims 1 to 13, is selected from

| Compound No. | Structure |
|---|---|
| 1 | 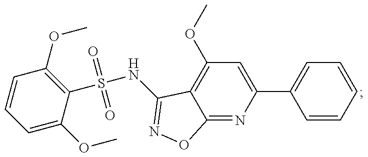 [SMILES: COc1cccc(OC)c1S(=O)(=O)Nc1noc2nc(-c3ccccc3)cc(OC)c12] |
| 2 | 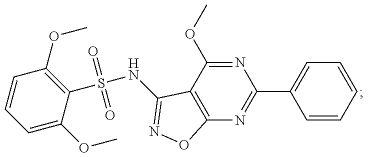 [SMILES: COc1cccc(OC)c1S(=O)(=O)Nc1noc2nc(-c3ccccc3)nc(OC)c12] |
| 3 | 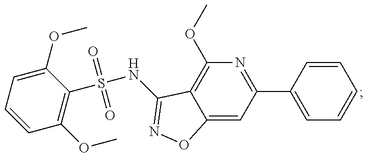 [SMILES: COc1cccc(OC)c1S(=O)(=O)Nc1noc2cc(-c3ccccc3)nc(OC)c12] |
| 4 | 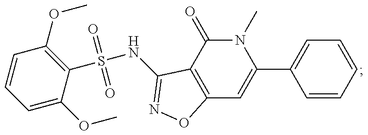 [SMILES: COc1cccc(OC)c1S(=O)(=O)Nc1noc2cc(-c3ccccc3)n(C)c(=O)c12] |
| 5 | 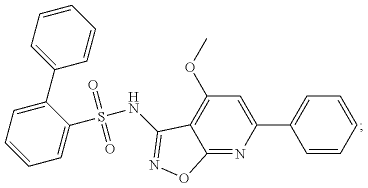 [SMILES: COc1cc(-c2ccccc2)nc2onc(NS(=O)(=O)c3ccccc3-c3ccccc3)c12] |
| 6 | 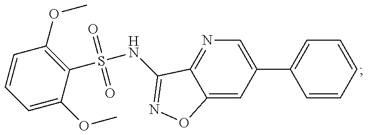 [SMILES: COc1cccc(OC)c1S(=O)(=O)Nc1noc2cc(-c3ccccc3)cnc12] |
| 7 | 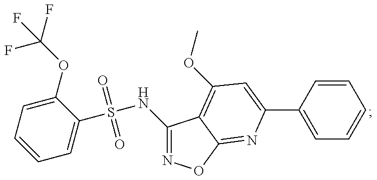 [SMILES: COc1cc(-c2ccccc2)nc2onc(NS(=O)(=O)c3ccccc3OC(F)(F)F)c12] |
| 8 | 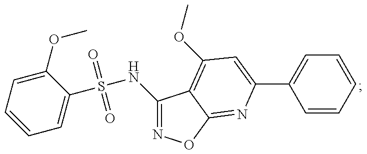 [SMILES: COc1ccccc1S(=O)(=O)Nc1noc2nc(-c3ccccc3)cc(OC)c12] |
| 9 | 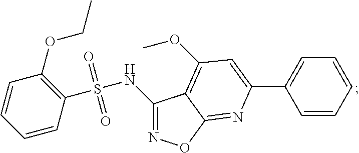 [SMILES: CCOc1ccccc1S(=O)(=O)Nc1noc2nc(-c3ccccc3)cc(OC)c12] |
| 10 | 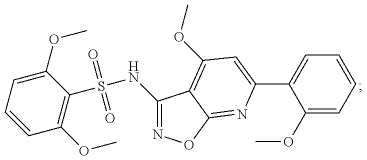 [SMILES: COc1ccccc1-c1cc(OC)c2c(NS(=O)(=O)c3c(OC)cccc3OC)noc2n1] |
| 11 | 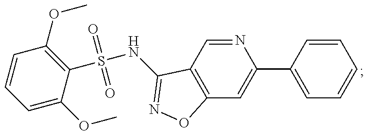 [SMILES: COc1cccc(OC)c1S(=O)(=O)Nc1noc2cc(-c3ccccc3)ncc12] |
| 12 | 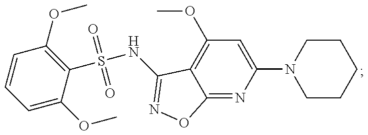 [SMILES: COc1cccc(OC)c1S(=O)(=O)Nc1noc2nc(N3CCCCC3)cc(OC)c12] |
| 13 | 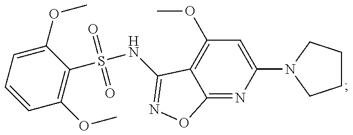 [SMILES: COc1cccc(OC)c1S(=O)(=O)Nc1noc2nc(N3CCCC3)cc(OC)c12] |
| 14 | 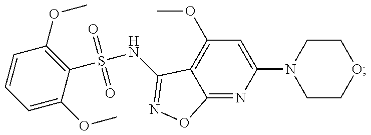 [SMILES: COc1cccc(OC)c1S(=O)(=O)Nc1noc2nc(N3CCOCC3)cc(OC)c12] |
| 15 | 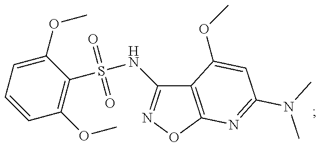 [SMILES: COc1cccc(OC)c1S(=O)(=O)Nc1noc2nc(N(C)C)cc(OC)c12] |
| 16 | 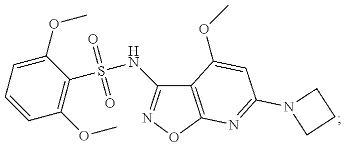 [SMILES: COc1cccc(OC)c1S(=O)(=O)Nc1noc2nc(N3CCC3)cc(OC)c12] |
| 17 | 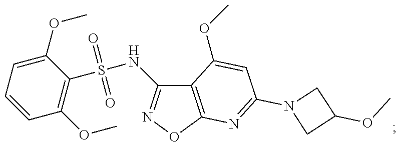 [SMILES: COc1cccc(OC)c1S(=O)(=O)Nc1noc2nc(N3CC(OC)C3)cc(OC)c12] |
| 18 | 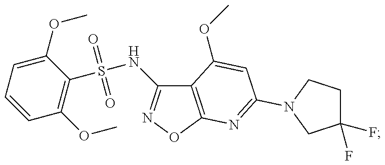 [SMILES: COc1cccc(OC)c1S(=O)(=O)Nc1noc2nc(N3CCC(F)(F)C3)cc(OC)c12] |
| 19 | 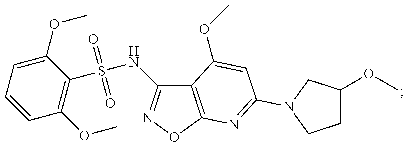 [SMILES: COc1cccc(OC)c1S(=O)(=O)Nc1noc2nc(N3CCC(OC)C3)cc(OC)c12] |
| 20 | 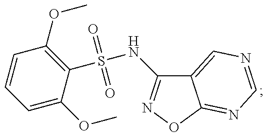 [SMILES: COc1cccc(OC)c1S(=O)(=O)Nc1noc2ncncc12] |
| 21 | 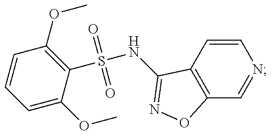 [SMILES: COc1cccc(OC)c1S(=O)(=O)Nc1noc2cnccc12] |
| 22 | 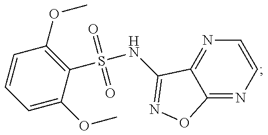 [SMILES: COc1cccc(OC)c1S(=O)(=O)Nc1noc2nccnc12] |
| 23 | 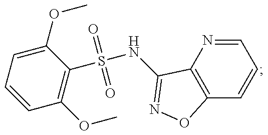 [SMILES: COc1cccc(OC)c1S(=O)(=O)Nc1noc2cccnc12] |
| 24 | 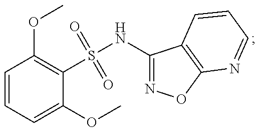 [SMILES: COc1cccc(OC)c1S(=O)(=O)Nc1noc2ncccc12] |
| 25 | 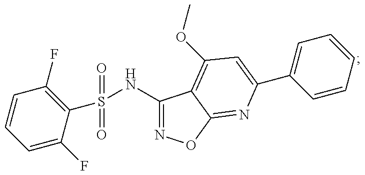 [SMILES: COc1cc(-c2ccccc2)nc2onc(NS(=O)(=O)c3c(F)cccc3F)c12] |
| 26 | 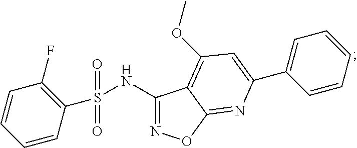 [SMILES: COc1cc(-c2ccccc2)nc2onc(NS(=O)(=O)c3ccccc3F)c12] |
| 27 | 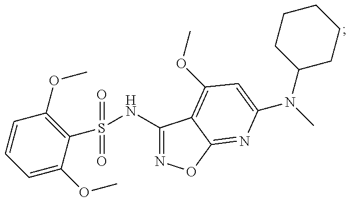 [SMILES: COc1cccc(OC)c1S(=O)(=O)Nc1noc2nc(N(C)C3CCCCC3)cc(OC)c12] |
| 28 | 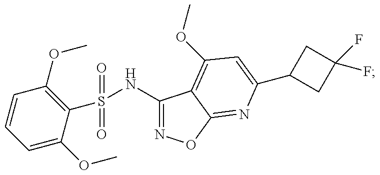 [SMILES: COc1cccc(OC)c1S(=O)(=O)Nc1noc2nc(C3CC(F)(F)C3)cc(OC)c12] |
| 29 | 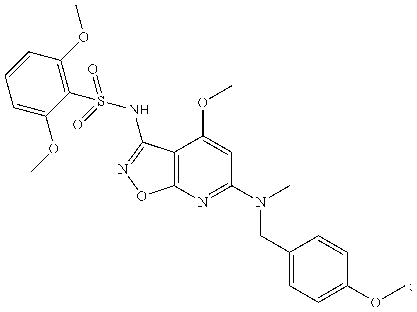 [SMILES: COc1ccc(CN(C)c2cc(OC)c3c(NS(=O)(=O)c4c(OC)cccc4OC)noc3n2)cc1] |
| 30 | 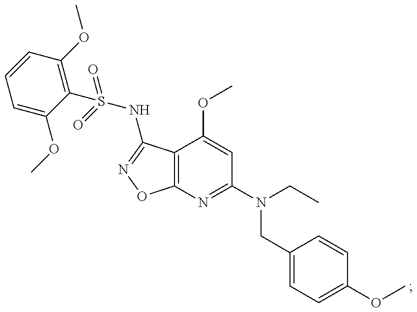 [SMILES: CCN(Cc1ccc(OC)cc1)c1cc(OC)c2c(NS(=O)(=O)c3c(OC)cccc3OC)noc2n1] |
| 31 | 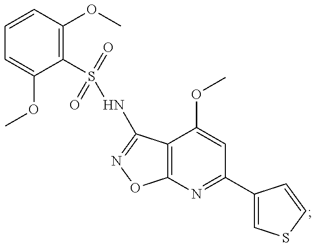 [SMILES: COc1cccc(OC)c1S(=O)(=O)Nc1noc2nc(-c3ccsc3)cc(OC)c12] |
| 32 | 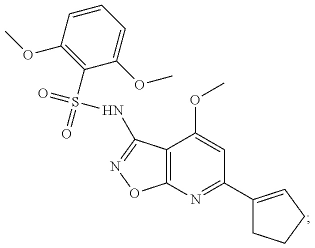 [SMILES: COc1cccc(OC)c1S(=O)(=O)Nc1noc2nc(C3=CCCC3)cc(OC)c12] |
| 33 | 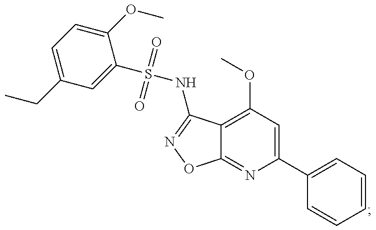 [SMILES: CCc1ccc(OC)c(S(=O)(=O)Nc2noc3nc(-c4ccccc4)cc(OC)c23)c1] |
| 34 | 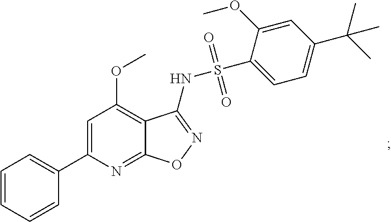 [SMILES: COc1cc(C(C)(C)C)ccc1S(=O)(=O)Nc1noc2nc(-c3ccccc3)cc(OC)c12] |
| 35 | 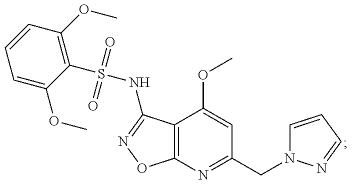 [SMILES: COc1cccc(OC)c1S(=O)(=O)Nc1noc2nc(Cn3cccn3)cc(OC)c12] |
| 36 | 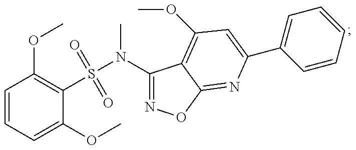 [SMILES: COc1cccc(OC)c1S(=O)(=O)N(C)c1noc2nc(-c3ccccc3)cc(OC)c12] |
| 37 | 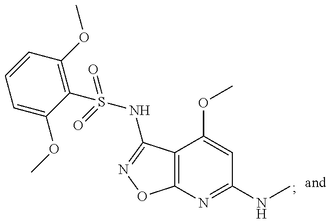 [SMILES: CNc1cc(OC)c2c(NS(=O)(=O)c3c(OC)cccc3OC)noc2n1] |
| 38 | 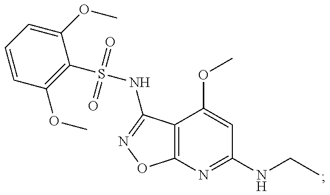 [SMILES: CCNc1cc(OC)c2c(NS(=O)(=O)c3c(OC)cccc3OC)noc2n1] |

or a pharmaceutically acceptable salt or a stereoisomer or a tautomer thereof.

15. A pharmaceutical composition comprising a compound of any one of claims 1 to 14 or a pharmaceutically acceptable salt or a stereoisomer or a tautomer thereof and a pharmaceutically acceptable carrier or excipient.

16. The pharmaceutical composition according to claim 15 for use as a medicament.

17. The pharmaceutical composition as claimed in claim 15 for use in the treatment of a disease or disorder mediated by the inhibition of KAT6A.

18. The pharmaceutical composition for use according to claim 17, wherein the disease or disorder is a cancer.

19. The pharmaceutical composition for use according to claim 18, wherein the cancer is selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer and virus-related cancer.

20. A compound according to any one of claims 1 to 14 or a pharmaceutically acceptable salt or a stereoisomer or a tautomer thereof, for use as a medicament.

21. A method of modulating KAT6A in a subject comprising administering to the subject, in need thereof, a therapeutically effective amount of a compound according to any one of claims 1 to 14 or a pharmaceutically acceptable salt or a stereoisomer or a tautomer thereof.

22. A method of treating a disease or disorder in a subject comprising administering to the subject, in need thereof, a therapeutically effective amount of a compound according to any one of claims 1 to 14 or a pharmaceutically acceptable salt or a stereoisomer or a tautomer thereof.

23. The method of claim 22, wherein the disease or disorder is a cancer.

24. The method of claim 23, wherein the cancer is selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductaladenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer and virus-related cancer.

25. Use of a compound according to any one of claims 1 to 14 or a pharmaceutically acceptable salt or a stereoisomer or a tautomer thereof, in the manufacture of a medicament for treating or preventing from a disease or disorder mediated by the inhibition of KAT6A.

26. The use according to claim 25, wherein disease or disorder is cancer selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductaladenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer and virus-related cancer.

27. Compound of any one of claims 1 to 14, for use in treating a disease or disorder mediated by KAT6A.

28. The compound for use according to claim 27, wherein disease or disorder is cancer selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer and virus-related cancer.
